SEQUENCE LISTING (9 sequences)

SEQ ID NO: 1 - DNA, 25 bp, Artificial Sequence
  hypoxanthine phosphoribosyltransferase (HPRT) - forward primer
ggcagtataatccaaagatggtcaa

SEQ ID NO: 2 - DNA, 25 bp, Artificial Sequence
  hypoxanthine phosphoribosyltransferase (HPRT) - reverse primer
gtctggcttatatccaacacttcgt

SEQ ID NO: 3 - DNA, 25 bp, Artificial Sequence
  hypoxanthine phosphoribosyltransferase (HPRT) - fluorescent probe labeled at 5′ with (FAM = 6-carboxy-fluorescein), at 3′ with (TAMRA = 6-carboxy-tetramethylrhodamine)
caagcttgctggtgaaaaggacccc

SEQ ID NO: 4 - DNA, 24 bp, Artificial Sequence
  canine HMGA2 - forward primer
agtccctccaaagcagctcaaaag

SEQ ID NO: 5 - DNA, 21 bp, Artificial Sequence
  canine HMGA2 - reverse primer
gccatttcctaggtctgcctc

SEQ ID NO: 6 - DNA, 24 bp, Artificial Sequence
  canine HMGA2 fluorescent probe labeled at 5′ with (FAM = 6-carboxy-fluorescein), at 3′ with (TAMRA = 6-carboxy-tetramethylrhodamine)
gaagccactggagaaaaacggcca

SEQ ID NO: 7 - DNA, 18 bp, Artificial Sequence
  GUSB - forward primer
tggtgctgaggattggca

SEQ ID NO: 8 - DNA, 20 bp, Artificial Sequence
  GUSB - reverse primer
ctgccacatggaccccattc

SEQ ID NO: 9 - DNA, 25 bp, Artificial Sequence
  GUSB - fluorescent probe labeled at 5′ with (FAM = 6-carboxy-fluorescein), at 3′ with (TAMRA = 6-carboxy-tetramethylrhodamine)
cgcccactactatgccatcgtgtgt

(19) United States
(12) Patent Application Publication
(10) Pub. No.: US 2014/0371237 A1
(43) Pub. Date: Dec. 18, 2014

(54) p19Arf, HMGA2 and MDM2 For Use in the Diagnosis and Treatment of Aberrant Cell Growth

(71) Applicant: University of Bremen, Bremen (DE)

(72) Inventors: Jörn Bullerdiek, Bremen (DE); Dominique N. Markowski, Bremen (DE)

(21) Appl. No.: 14/453,090

(22) Filed: Aug. 6, 2014

Related U.S. Application Data
(62) Division of application No. 13/578,010, filed on Oct. 31, 2012.

(30) Foreign Application Priority Data
Feb. 9, 2010 (EP) 10 001 307.7
May 4, 2010 (EP) 10 004 715.8

(51) Int. Cl. A61K 31/496; A61K 45/06

(52) U.S. Cl. CPC A61K 31/496; A61K 45/06
USPC 514/254.05

(57) ABSTRACT

Provided are novel methods and compositions for the diagnosis, prognosis and treatment of leiomyomas, in particular uterine leiomyoma (UL). In addition, methods of identifying anti-tumor agents are described. Furthermore, novel methods and compositions are provided for the treatment of diseases characterized by an aberrant growth of mesenchymal stem cells and their descendants and for the treatment of obesity are disclosed.

### CROSS-REFERENCE TO RELATED APPLICATIONS

This application is a Divisional Application of and claims priority to U.S. application Ser. No. 13/578,010 filed Aug. 9, 2012, which application is a U.S. National Stage application of International Application No. PCT/EP2011/000596, filed Feb. 9, 2011, which application relies on the disclosure of and claims priority to European Patent Application No. 10 001 307.7, filed Feb. 9, 2010, and European Patent Application No. 10 004 715.8, filed May 4, 2010, the disclosures of which are hereby incorporated by reference herein in their entireties.

### REFERENCE TO SEQUENCE LISTING SUBMITTED IN COMPUTER READABLE FORM

The present application contains a Sequence Listing which has been submitted in ASCII format via EFS-Web and is hereby incorporated by reference herein in its entirety. The ASCII file was created Aug. 6, 2014 and named MULRDIVsequence_ST25, which is 5.69 kilobytes in size and which is identical to the paper copy filed with this application.

### FIELD OF THE INVENTION

The present invention is generally concerned with the expression, detection of gene products and modulation of activity of the genes p19^{Arf}, HMGA2 and MDM2 for use in the diagnosis of diseases associated with aberrant cell growth and with the provision of novel means in treatment of said diseases. In a first aspect the present invention relates to a method of diagnosing whether a subject has, or is at risk for developing leiomyomas, in particular uterine leiomyoma (UL) comprising measuring the level of p19^{Arf} and HMGA2 in a test sample derived from a UL, wherein increased levels of p19Arf and/or MDM2 and/or their gene transcripts, respectively, and an increased level of HMGA2 and/or its gene transcript, respectively, in the test sample relative to the level of levels compared to a control sample is indicative of a rapidly growing leiomyoma that is sensitive. Furthermore, a novel therapy of UL is provided by use of an MDM2 inhibitor for use in the treatment of a patient in need thereof. In a further aspect the present invention generally relates to MDM2 inhibitors for use in the treatment of diseases characterized by an aberrant growth of cells and tissues of mesenchymal origin.

Several documents are cited throughout the text of this specification. The contents of all cited references (including literature references, issued patents, published patent applications as cited throughout this application and manufacturer's specifications, instructions, etc) are hereby expressly incorporated by reference; however, there is no admission that any document cited is indeed prior art as to the present invention.

### BACKGROUND OF THE INVENTION

Uterine leiomyomas are benign smooth muscle tumors with an extremely high prevalence making them the most frequent gynecological tumors at all. Based on histology to detect small tumors and on ultrasound studies it has been estimated that up to approximately 70-80% of women in their reproductive age have one or more leiomyomas (Cramer and Patel, 1990; Baird et al., 2003). Symptomatic leiomyomas are a major public health problem and one of the leading causes for hysterectomy accounting for approximately 30% of all hysterectomies in the United States annually (Sandberg, 2005). Compared to this enormously high prevalence of fibroids still relatively little is known about their pathogenesis and etiology. Though numerous hypotheses have been put forward to explain the development of fibroids (for review see Sandberg, 2005) their monoclonal origin suggests mutations as the leading cause of abnormal proliferation of smooth muscle cells. Thus, pathogenetically, uterine leiomyomas (UL) can be interpreted as the result of a monoclonal abnormal proliferation of myometrial cells.

As to consistent mutations observed in considerable percentages of fibroids some groups of recurrent chromosomal aberrations have been identified (for review see Ligon and Morton, 2000; Sandberg, 2005). A frequent subgroup of uterine leiomyomas (UL) is characterized by clonal translocations of chromosomal region 12q14˜15 targeting HMGA2 (Schoenmakers et al., 1995) and leading to its drastic upregulation (Gross et al., 2003; Klemke et al., 2009). Interestingly, UL of this type show an increased size compared to those without detectable cytogenetic deviations (Rein et al., 1998; Hennig et al., 1999). Although it is tempting to speculate that the overexpression of HMGA2, a protein abundantly expressed in stem cells and linked to their self-renewal (Li et al., 2006; Li and Dröge, 2007; Nishino et al., 2008) accounts for that enhanced growth potential, the exact mechanisms by which HMGA2 can influence UL growth still remain to be resolved. Recently a link between HMGA2 and the Ink4a/Arf locus in somatic stem cells has attracted a lot of interest. Oncogene-induced senescence (OIS) is a frequent phenomenon in premalignant lesions that leads to a growth arrest mainly by the activation of two potent growth-inhibitory pathways, i.e. p16^{Ink4a} and p19^{Arf}. The relevance of OIS for the development of UL has not been addressed so far in detail but HMGA2, encoded by a major target gene of recurrent chromosomal abnormalities in UL has been implicated recently in the repression of the Ink4a/Arf locus.

Expression of that locus is associated with the control of cellular senescence in many cell types and being part of the lin28-let-7-HMGA2 axis the Ink4a/Arf locus was found to be repressed by HMGA2 (Nishino et al., 2008). Accordingly, it can be speculated that repression of the Ink4a/Arf locus by the abundance of HMGA2 accounts for the larger size of UL with HMGA2 rearrangements compared to those without that mutation (Rein et al., 1998; Hennig et al., 1999). By immunohistochemistry, p16^{Ink4a} positivity was found more often in leiomyosarcomas than in leiomyomas where it appears to be restricted to single cases only (Atkins et al., 2008; Lee et al., 2009) but to the best of the inventor's knowledge studies addressing the Ink4a/Arf expression in the different genetic types of UL have not been performed yet. Nevertheless, p19^{Arf} is known to stabilize p53 by its regulation of MDM2 which in turn promotes p53 degradation (Zhang et al., 1998; Meek, 2009). Thus, the lack of immunoreactivity for p53 seen in almost all leiomyomas (Lee et al., 2009) may exclude indirectly the presence of Arf in a substantial number of leiomyomas thus confirming the hypothesis outlined above.

Although only a minority of the leiomyomas become symptomatic the presence of symptomatic leiomyomas is still the leading cause for hysterectomy worldwide. Despite their high prevalence the treatment options besides surgical removal by hysterectomy or tumor enucleation are still limited. Treatment by GnRH agonists as well as antagonists can induce shrinkage of fibroids but re-growth of the tumors usually occurs after termination of the therapy [3, 4]. Thus, intervention at the hormonal level is as a rule only recommended to reduce tumor size pre-operatively [5]. Another alternative represents embolization of the fibroids but the recurrence of myoma-related symptoms is not a rare finding after that treatment as well [6]. Thus, therapies aimed at permanent shrinkage of the fibroids still remain a challenge.

The above-mentioned problems are solved by the embodiments characterized in the claims and described further below.

### SUMMARY OF THE INVENTION

In a first row of experiments a series of UL was investigated with 12q14˜15 rearrangements and with other aberrations, respectively, for their expression of both genes of the locus. Unexpectedly, no inverse relationship was noted but an overexpression of one of the two genes encoded by the Cdkn2a locus, i.e. Arf, could be shown in uterine leiomyomas compared to myometrial tissue.

Accordingly, the methods and uses as well as medical treatments of the present invention as characterized in the claims are based on the experimental observations described herein. In this context, the present invention also pertains to a pharmaceutical composition for treating a subject having, or being at risk for developing and growth of UL, said composition comprising a compound capable of agonizing p19^{Arf} and/or antagonizing HMGA2 or preferably antagonizing MDM2; and optionally a pharmaceutically acceptable carrier.

For example, the growth of UL may be inhibited by administering to the subject an effective amount of p19^{ARF} or a mimetic thereof, and optionally p53 as described in US patent application US 2003/176350 A1. The management of uterine leiomyomas is reviewed for example by Lefebvre et al., J. Obstet. Gynaecol. Can. 25 (2003), 396-418; quiz 419-422, and includes medical treatments, conservative treatments of myolysis, selective artery occlusion, and surgical alternatives including myomectomy and hysterectomy. For example, present invention envisages the use of a vaginally administrable tablet for treating leiomyomata, leiomyoma, myoma, uterine fibroids, endometriosis, adenomyosis and other related disorders which have been diagnosed in accordance with the present invention. The tablet may comprise mifepristone, at least one non-effervescent excipient or diluent, and at least one effervescent excipient as described in international application WO2009/037704 A1. In addition, or alternatively, GnRH agonists and antagonists may be used in the preoperative therapy of UL; see for review, e.g., De Falco et al., in Minerva Ginecol. 58 (2006), 553-560.

Inhibitors of HMGA2 binding to DNA such as netropsin and methods of screening such compounds are known in the art and described, for example by Miao et al. in Anal. Biochem. 374 (2008), 7-15.

Antagonists and inhibitors of the oncogenic activity of the protein MDM2 are also well known to the person skilled in art; see, e.g., international applications WO 1997/009343 and WO 2003/095625. Useful inhibitors include the class of 1,4-benzodiazepines, which act as inhibitors of MDM2-p53 interactions. Further inhibitors of a p53-MDM2 interaction as well as pharmaceutical compositions comprising said inhibitors are known in the art and described in, e.g., international applications WO 2007/107543, WO 2007/107545 and WO 2009/156735. Accordingly, as exemplified herein based on in vitro cultures of cells from uterine fibroids antagonizing MDM2 by appropriate inhibitors offers a novel way to treat uterine leiomyomas by inducing senescence as well as apoptosis within the fibroids (see Brown et al., Nat. Rev. Cancer 9 (2009), 862-873: “Awakening guardian angels: drugging the p53 pathway” for review).

In a second series of experiments additional tissue samples from UL (see Table 1 in FIGS. 20A and 20B) were investigated confirming an overexpression of Arf in uterine leiomyomas compared to myometrial tissue. Further antagonists and inhibitors of the expression, respective oncogenic activity of the protein MDM2 were tested as well, confirming the induction of senescence and apoptosis in the UL-cells and the general usefulness of MDM2 antagonists, on the basis of their common mode of action in the therapy of UL. Furthermore, in addition to cells and tissue from uterine leiomyomas, cells from another tissue of mesenchymal origin, i.e. adipose-tissue derived stem cells (ADSCs) were treated with an MDM2-inhibitor. Comparable to the effect on UL cells, said treatment led to an decreased self-renewal capacity of these cells as well, pointing at a general usefulness of the methods and compounds of the present invention in the treatment of diseases characterized by an aberrant growth of mesenchymal stem cells and their descendants, wherein the diseases are selected from the group comprising endometriosis, adenomyosis, endometrial hyperplasia, leiomyoma, lipoma, hamartoma of the lung, fibroadenoma of the breast, adenoma of the salivary gland, and aggressive angiomyxomas. Accordingly, obesity, as a disorder characterized by an overgrowth of the adipose tissue may be treated according to the methods of the present invention as well.

A more complete understanding can be obtained by reference to the following specific examples which are provided herein for purposes of illustration only and are not intended to limit the scope of the invention.

### BRIEF DESCRIPTION OF THE DRAWINGS

FIG. 1 is a graph showing relative quantification of the Arf expression in uterine leiomyomas and myometrial tissues. Black bars (bars 1, 4-6, 8-10): myometrium; white bars (bars 2, 12, 13, 17, 19, 20, 27): UL with normal karyotype; red bars (bars 3, 11, 18, 21-24, 28-30): UL with 12q14˜15 aberrations; blue bars (bars 7, 14-16, 25): UL with other clonal cytogenetic aberrations. ADSCs (6th passage) served as calibrator (grey bar (bar 26); expression: 1).

FIG. 2 is a graph showing correlation between the relative HMGA2 expression (x-axis) and the relative Arf expression (y-axis) in myometrium (), UL with 12q14˜15 aberrations ( [symbol] ), UL with a normal karyotype (□) and UL with other clonal cytogenetic aberrations ( [symbol] ) ADSCs (6^{th} passage) served as calibrator (expression: 1).

FIG. 3 is a graph showing correlation between the relative Arf expression (y-axis) and the relative MDM2 expression (x-axis) in uterine leiomyomata with 12q14˜15 aberrations () and uterine leiomyomata with an apparently normal karyotype (⋄). Native myometrial tissue served as calibrator (expression: 1). Triangles: myometrial tissue.

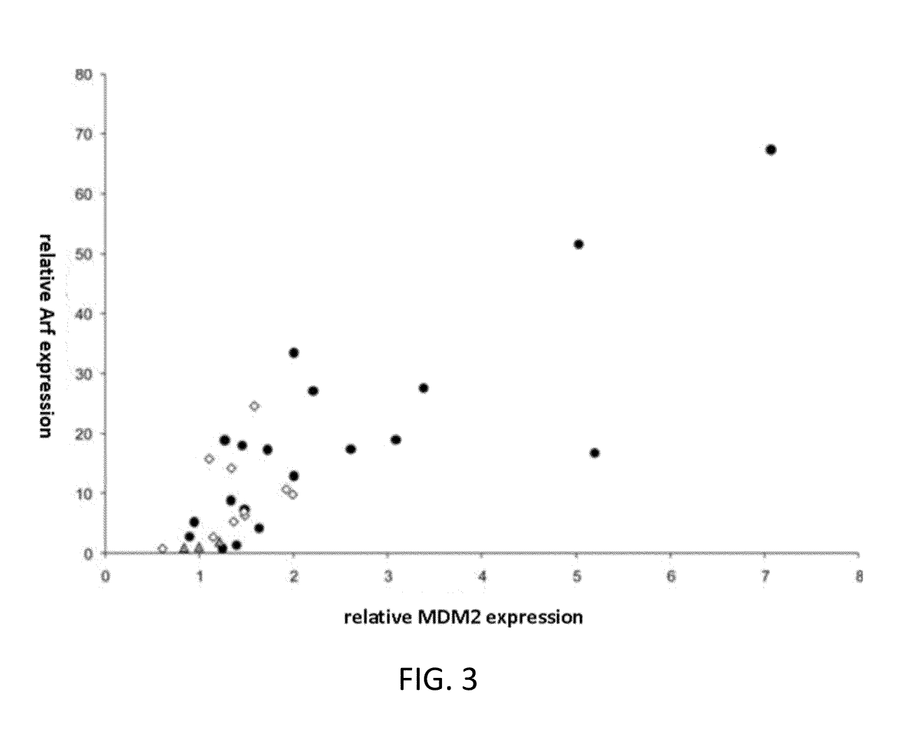

FIG. 4 is a graph showing correlation between the relative Arf expression (x-axis) and the relative Ki-67 expression (y-axis) in uterine leiomyomata with 12q14˜15 aberrations () and uterine leiomyomata with an apparently normal karyotype (⋄). Native myometrial tissue served as calibrator (expression: 1).

FIGS. 5A-5F are microscopic images showing in situ β-galactosidase staining increases after treatment of fibroids cells by treatment with the MDM2 antagonist nutlin-3. FIG. 5A: control, UL cells grown for 24 hours, FIG. 5B: control, UL cells grown for 72 hours, FIG. 5C: UL cells grown for 24 hours with 30 μM nutlin-3, FIG. 5D: UL cells grown for 72 hours with 30 μM nutlin-3, FIG. 5E: UL cells grown for 24 hours with 50 μM nutlin-3, FIG. 5F: UL cells grown for 72 hours with 5 μnM nutlin-3.

FIGS. 6A and 6B are graphs showing that in vitro treatment of UL cells by the MDM2 antagonist nutlin-3 for 24 and 72 hours, respectively, influences important growth parameters. For all analyses control was set 100%. Expression of p21, beta-galactosidase (β-gal), Ki-67, and BAX was determined by qRT-PCR. The percentage of beta-galactosidase positive cells (total number of cells checked: control, 24 h: 654; 30 μM nutlin-3, 24 h: 471; 50 μM nutlin-3, 24 h: 546; control, 72 h: 1446; 30 μM nutlin-3, 72 h: 222; 50 μM nutlin-3, 72 h: 510) and mitoses were analyzed microscopically. Statistical significances have been tested for the qRT-PCR data and the results given by asterisks (*, p<0.05; **, p<0.01; ***, p<0.001).

FIG. 7 is a graph showing a highly significant correlation between the relative BAX expression (x-axis) and the relative p19Arf expression (y-axis) in myometrium ( [symbol] ), UL with 12q14˜15 aberrations (), and UL with a normal karyotype (⋄). Myometrial tissue served as calibrator (expression: 1).

FIG. 8 is a graph showing the correlation between the relative BAX expression (x-axis) and the relative p21 expression (y-axis) in myometrium ( [symbol] ), UL with 12q14˜15 aberrations (), and UL with a normal karyotype (⋄). Myometrial tissue served as calibrator (expression: 1).

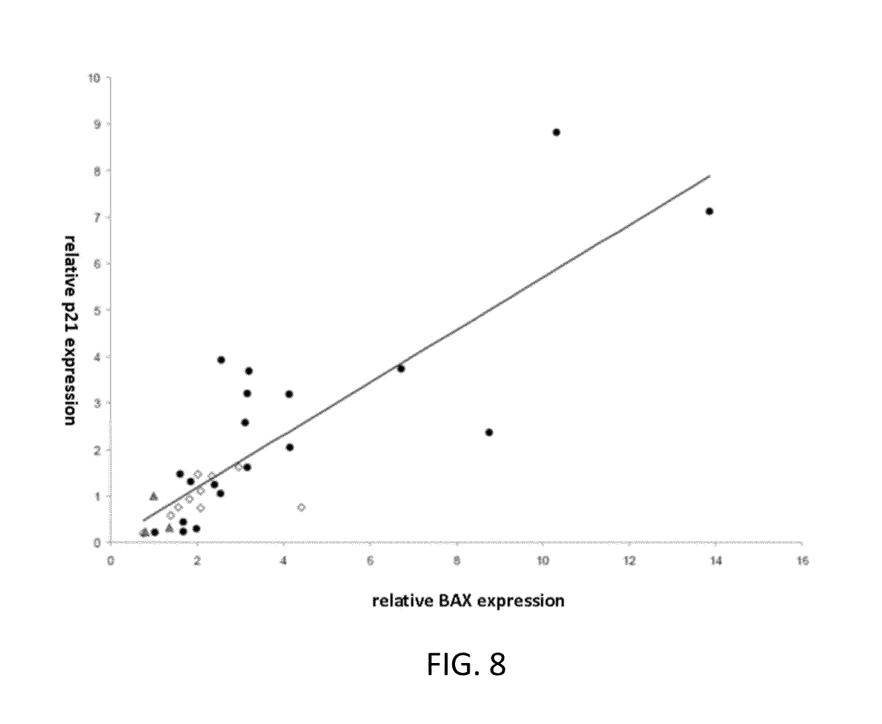

FIG. 9 is a schematic diagram showing a model delineating the interaction of key elements of a senescence pathway active in uterine leiomyomas. The model is based on the finding that leiomyomas express significantly higher levels of p19Arf mRNA than myometrial tissue. Most likely due to the repression of MDM2 followed by the stabilization of TP53, this corresponds to an increase of CDKN1A expression, the latter being a direct target of transcriptional activation by TP53. In turn, a negative feedback loop between TP53 and p19Arf (Robertson and Jones, Mol. Cell. Biol. 18 (1998), 6457-6473) and a positive feedback loop between TP53 and MDM2 (Zhang et al., 1998; Meek, 2009) exist. The higher expression of p19Arf in UL with 12q14_{—}15 rearrangements compared with those of other cytogenetic subtypes suggests that HMGA2 may behave like a classical oncogene inducing p19Arf-driven oncogene-induced senescence. Although it is not clear how HMGA2 can influence p19Arf transcription, one hypothetical way might be via its possible interaction with pRB1 (Fedele et al., Cancer Cell 9 (2006), 459-471) followed by the activation of p19Arf by E2F1 (cf. Komori et al., EMBO J. 24 (2005), 3724-3736). Genes investigated in this study are highlighted by pink background, and alternative routes of possible minor relevance in fibroids are represented by hatched lines.

FIGS. 10A-10C are graphs showing tissue explants taken from uterine fibroids display sensitivity to nutlin-3 as revealed by the increased expression of p21, BAX, and decreased expression of Ki-67 mRNA after 72 h incubation with nutlin-3 compared to the controls. Expression in the control explants (white bars, no treatment) was always set 100%. Ordinate: % change of expression compared to control. Hatched bars: 3 μM nutlin-3; grey bars: 10 μM nutlin-3. For tumor numbers below each group of bars rf. to Tab. 1. FIG. 10A: p21, FIG. 10B: BAX, FIG. 10C: Ki-67. Statistically significant increases (p21 and BAX) or decreases (Ki-67) are given by asterisks (**, p<0.01; ***, p<0.001).

FIG. 11A is an image of a Western Blot analysis of p53 of explants from an UL (case 700-1) treated with 30 μM and 50 μM nutlin-3 for 72 h showing a concentration-dependent increase of the amount of p53. Lane 1: 50 μM nutlin-3, lane 2: 30 μM nutlin-3, lane 3: control without nultin-3, lane 4: marker SeeBlue Plus2 Pre-Stained Standard (Invitrogen, Karlsruhe, Germany) (left to right).

FIG. 11B is a graph showing p53 protein expression determined after immunoblotting (c.f. A) by ImageJ (as described in the materials and methods section) against beta-actin. Control was set 100%. Ordinate: % change of p53 expression compared to control.

FIG. 12 is a graph showing Leiomyomas usually express higher levels of p14Arf than matching myometrium. Columns within each row give the relative expression of p14Arf mRNA in myometrium (black columns) and matching fibroids (gray columns) from one patient each as revealed by qRT-PCR. Number below each row corresponds to the patient's lab no. The corresponding leiomyomas are depicted in a numerical order (cf. FIGS. 20A and 20B). Ordinate gives relative expression of p14Arf.

FIGS. 13A-13D are graphs showing as a rule, fibroids display higher nutlin-3 sensitivity than matching myometrium. Explants from five fibroids from three patients were checked for their nutlin-3 sensitivity after incubation with 3 μM or 10 μM nutlin-3 respectively, for 72 hours. As an indicator for sensitivity the expression of BAX (FIG. 13A, FIG. 13B) and p21 (FIG. 13C, FIG. 13D) mRNA was determined by qRT-PCR. Myometrium (black columns) was always set 100% and the expression of the corresponding fibroids (gray columns) refers to that value. Numbers below each row indicate the patient's lab no. (cf. table in FIGS. 20A and 20B). The corresponding leiomyomas are depicted in numerical order. Statistically significances are given by asterisks (*, p<0.05; **, p<0.01; ***, p<0.001).

FIGS. 14A-14L are graphs showing after incubation with nutlin-3 for six days, an increased expression of p21 as well as BAX and a decreased expression of Ki-67 compared to the controls were noted. Myometrium (columns indicated with “C” each; not treated) was always set 100% and the expression of the corresponding fibroids refers to that value. (FIGS. 14A-D): expression of p21 mRNA, (FIGS. 14E-H): expression of BAX mRNA, (FIGS. 14I-L): expression of Ki-67 mRNA. For sample numbers refer to Tab. 1. Statistically significant increases (p21, and BAX) or decreases (Ki-67) are given by asterisks (*, p<0.05; **, p<0.01; ***, p<0.001). n.d.: not detectable.

FIGS. 15A-15C are graphs showing increase of the expression of p21, BAX respective Ki-67 mRNA after in vitro treatment of leiomyoma cells with siRNA (SIH900207ABCD). (FIG. 15A): Increase of the expression of p21 after said treatment of leiomyoma cells. Significant differences (p<0.001) to the negative control (Negativ siRNA) were found for the four different siRNAs used. (FIG. 15B): Increase of the expression of BAX mRNA after said treatment of leiomyoma cells. Except for A and D significant differences to the negative control (negative siRNA) were found for siRNAs B (p<0.001) and C (p<0.01). (FIG. 15C): Decrease of the expression of Ki-67 mRNA after in vitro treatment of leiomyoma cells with siRNA (SIH900207ABCD). Significant differences (p<0.001) to the negative control (negative siRNA) were found for the four different siRNAs used.

FIGS. 16A-16D are graphs showing in vitro treatment of leiomyoma cells with RITA leads to (FIG. 16A): An increase of the expression of p21 mRNA. (FIG. 16B): An increase of the expression of β-galactosidase mRNA. (FIG. 16C): An increase of the expression of BAX mRNA. (FIG. 16D): A decrease of the expression of Ki-67 mRNA. Columns indicated with “C” are non treated controls with the respective expression set to 1. The expression of the corresponding cells refers to that value.

FIGS. 17A and 17B are graphs showing in vitro treatment of explants with RITA leads to (FIG. 17A): an increase of the expression of p21 mRNA and (FIG. 17B): of the expression of BAX mRNA as well. Columns indicated with “C” are non treated controls with the respective expression set to 1. The expression of the corresponding explants refers to that value.

FIGS. 18A and 18B are graphs showing in vitro treatment of leiomyoma cells with tenovin-1 leads to (FIG. 18A): an increase of the expression of p21 mRNA after. Significant differences (p<0.01) to the negative controls (24 h C and 72 h C, respectively) were noted with both concentrations used; (FIG. 18B): an increase of the expression of BAX mRNA. Significant differences to the negative controls (24 h C and 72 h C, respectively) were noted for 24 hours with 3 μM and 10 μM tenovin-1 (p<0.001) and 72 hours with 10 μM tenovin-1 (p<0.01).

FIGS. 19A and 19B are graphs showing in vitro treatment of mesenchymal stem cells with nutlin-3 leads to FIG. 19A: Decrease of the expression of HMGA2 and FIG. 19B: Increase of the expression of p21 mRNA. Dark and pale stained bars represent two different donors in each diagram. Asterisks indicate differences; **, p<0.01; ***, p<0.001.

FIGS. 20A and 20B are tables showing age of the patients, tumor size and karyotype of the leiomyomas investigated. Karyotypes are described according to [18].

### DETAILED DESCRIPTION OF THE INVENTION AND EXAMPLES

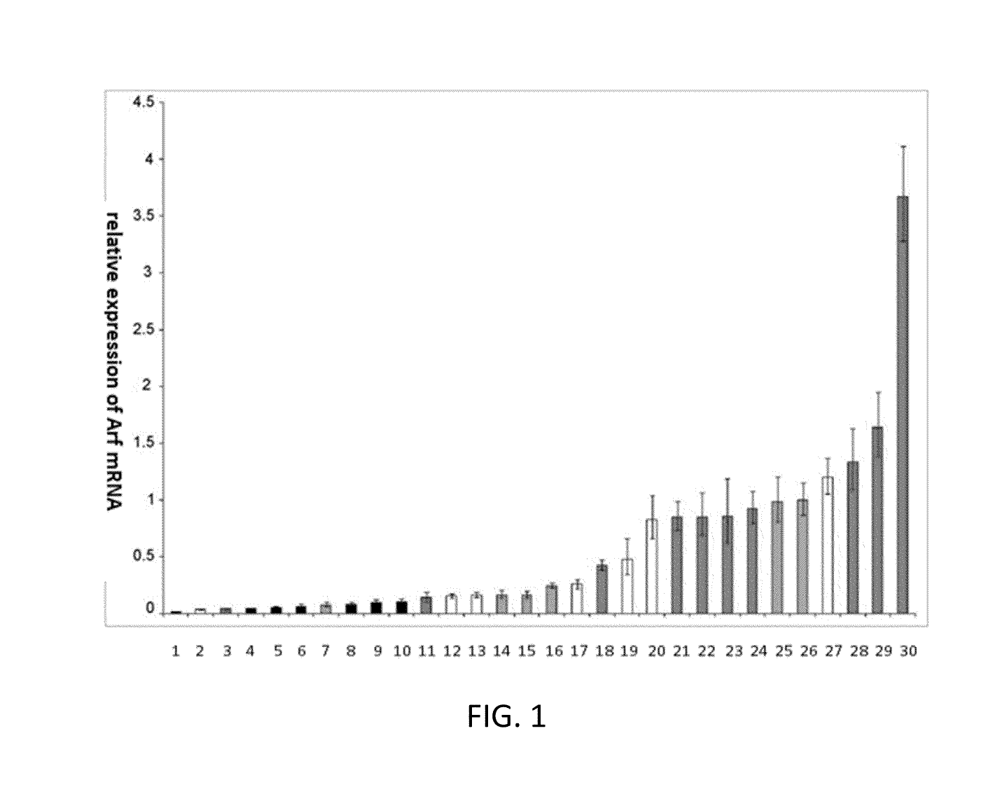

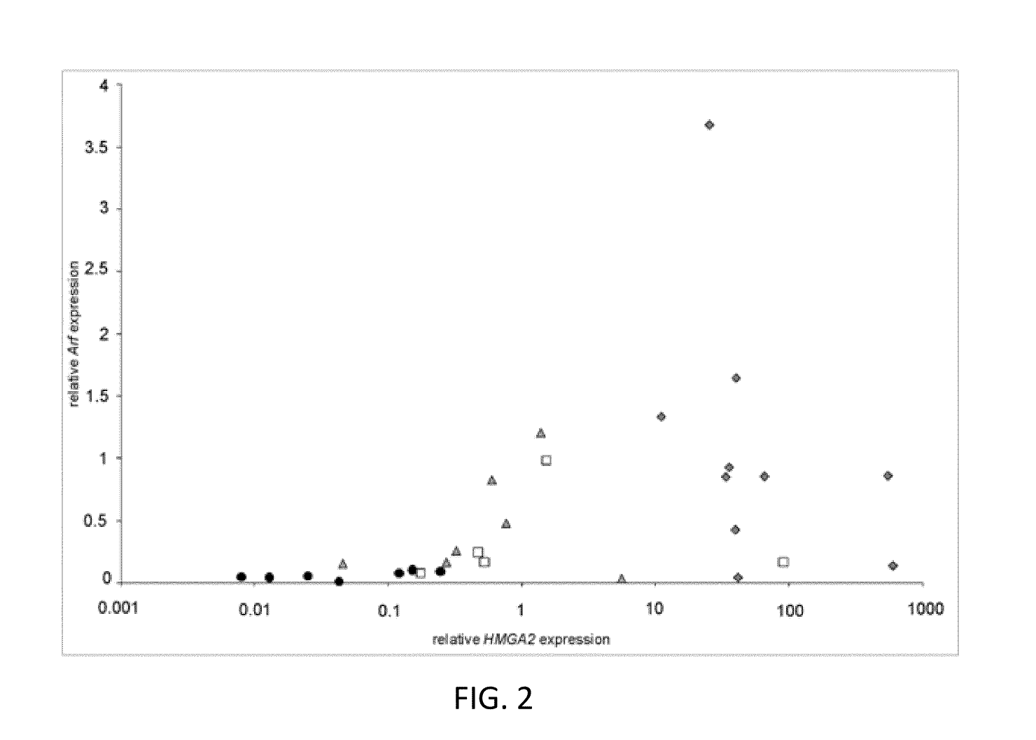

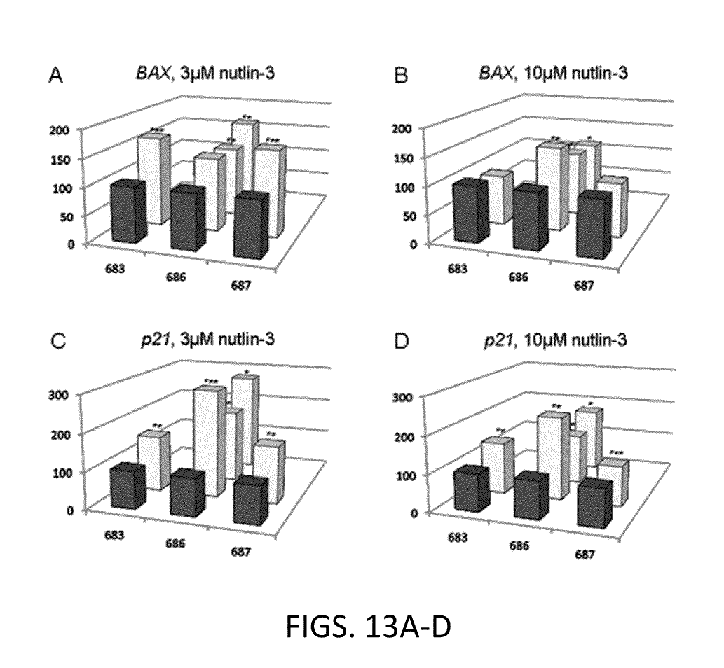

In the first (see Examples 1 to 3) and second series of experiments (Example 4) it could surprisingly be shown that in leiomyoma development the overexpression of p19^{Arf}/p14^{Arf} (the terms Arf, p14, p19, p14Arf; p19Arf will be used interchangeably herein) drives a negative feedback-loop between p53 and MDM2 that governs the fate of the individual fibroid. Compared to matching myometrial tissue the myomas display a significantly higher expression of one of the genes of the senescence associated Ink4a/Arf locus, i.e. p14^{Arf [}7]. It is not clear yet whether this elevated expression solely results from an enhanced proliferative activity of the fibroid compared to its tissue of origin or if the same oncogenic stimuli triggering the leiomyoma growth do simultaneously stimulate p14^{Arf} as an oncogene-induced senescence-like mechanism. Nevertheless, it is shown herein that generally UL express significantly higher levels of p19^{Arf} but not p16^{Ink4a} mRNA than myometrium; see Example 1 with the corresponding FIG. 1 and Example 4/FIG. 12. Moreover, while on average leiomyomas showing clonal rearrangements of 12q14˜15 expressed higher levels of p19^{Arf} mRNA than tumors of other cytogenetic subgroups there was no linear correlation with expression of HMGA2; see Example 1 and FIG. 2. However, leiomyomas with 12q14˜15 rearrangements are known to overexpress HMGA2 (Gross et al., 2003; Klemke et al., 2009) and also in this series its expression exceeded that of the fibroids with other cytogenetic subgroups in nearly all cases. From these data the inventors have concluded that HMGA2 in a dose dependent manner can exert opposite effects on the expression of p19^{Arf} that as a causal factor may explain the growth arrest seen in the majority of these highly frequent benign tumors. Therefore, in one particular embodiment the present invention relates to a method to diagnose the growth potential of an uterine leiomyoma (UL) comprising measuring the level of p19^{Arf} and HMGA2 in a test sample derived from a UL, wherein (a) an increased level of p19^{Arf} and (b) a decreased or an increased level of HMGA2 in the test sample relative to the level of HMGA2 compared to a control sample is indicative of a rapidly growing leiomyoma.

In a further embodiment the present invention relates to a pharmaceutical composition for treating a subject having, or being at risk for developing and growth of UL, said composition comprising a compound capable of agonizing p19^{Arf} and/or antagonizing HMGA2 and/or MDM2; and optionally a pharmaceutically acceptable carrier, wherein preferably the UL of the subject has been diagnosed in accordance with the method to diagnose the growth potential of an UL as defined above.

The present invention further relates to a method of identifying an anti-proliferative agent, comprising providing a test agent to a cell over-expressing p19^{Arf} and HMGA2, wherein a decrease in the level of overexpression of HMGA2 and/or an increase in the expression of p19^{Arf} relative to a control cell without being subjected to the test compound, is indicative of the test compound being an anti-proliferative agent, wherein preferably the test cell and control cell are derived from a sample of UL with or without chromosomal rearrangements of 12q14˜15.

Furthermore, in one embodiment the present invention provides a kit useful in a method as defined above, comprising one or more reagents for detecting the expression of p19^{Arf} and/or HMGA2, wherein preferably the reagents comprise an antibody or a nucleic acid, preferably further comprising primers to quantify the transcript of p19^{Arf} and/or HMGA2.

Naturally, any embodiment which is described or can be derived from the preceding description and examples and the appended figures is encompassed as well within the scope of the present invention.

The findings obtained in accordance with the present invention suggest that in vitro senescence of leiomyoma cells is controlled by the HMGA2-p19Arf axis. Thus, a similar mechanism seems to account for the in vivo aging of stem cell populations as well as for in vitro senescence of cells from uterine fibroids. Therefore, it may be assumed that in vivo senescence contributes to the growth control of UL as well. It was demonstrated, e.g. in Example 1 with the corresponding FIG. 1 and in Example 4/FIG. 12, that uterine leiomyomas express significantly higher levels of p19Arf than myometrial tissue and that in leiomyomas delicate balances along the HMGA2-p19Arf-MDM2-p53-p21 axis seem to exist that dictate the fate of the individual fibroids. Thus, in one embodiment the present invention relates to the use of the expression of Arf transcripts and translation products to predict the growth potential of uterine fibroids.

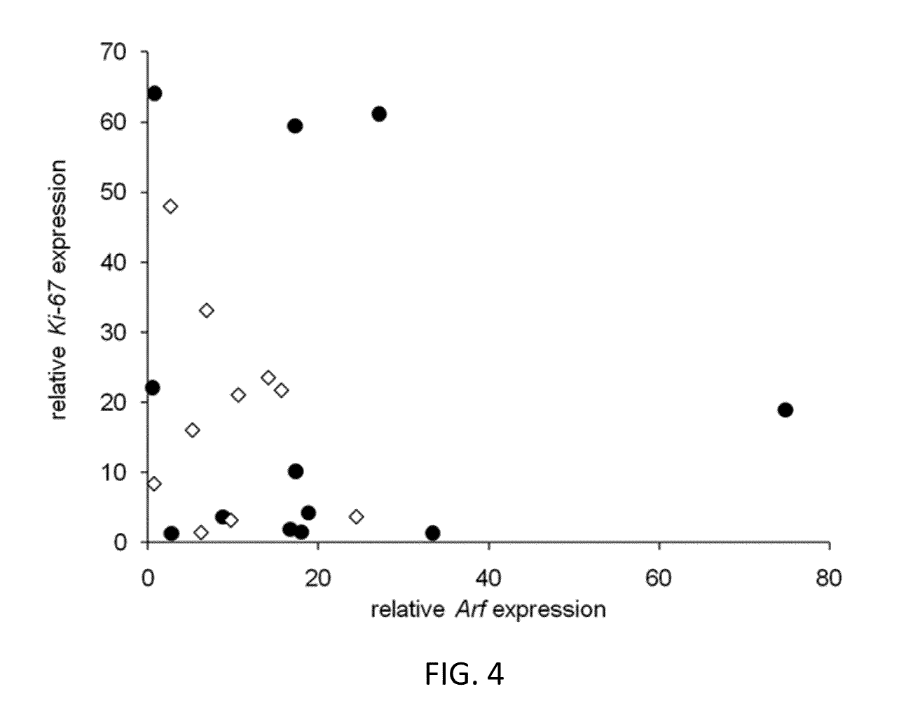

Ki-67 protein is strictly associated with cell proliferation, which is present during all active phases of the cell cycle (G1, S, G2 and mitosis) and is absent from the G₀ cells. Its expression ist therefore widely used as prognostic tools in cancer diagnostics (Bullwinkel et al., Journal of Cellular Physiology 206 (2006), 624-635; Scholzen and Gerdes, Journal of Cellular Physiology 182 (2000), 311-322) and as shown in Example 2 and FIG. 4, may be used according to methods of present invention as well. Thus, in another embodiment the present invention also relates to the use of a combination of the expression of Arf and Ki-67 transcripts and translation products to predict the growth potential of uterine fibroids.

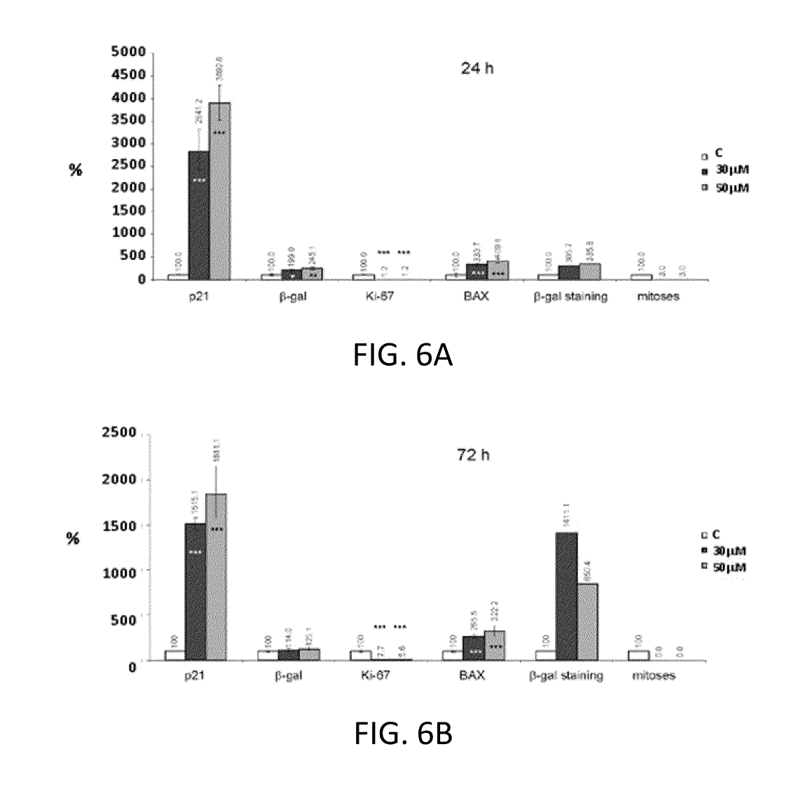

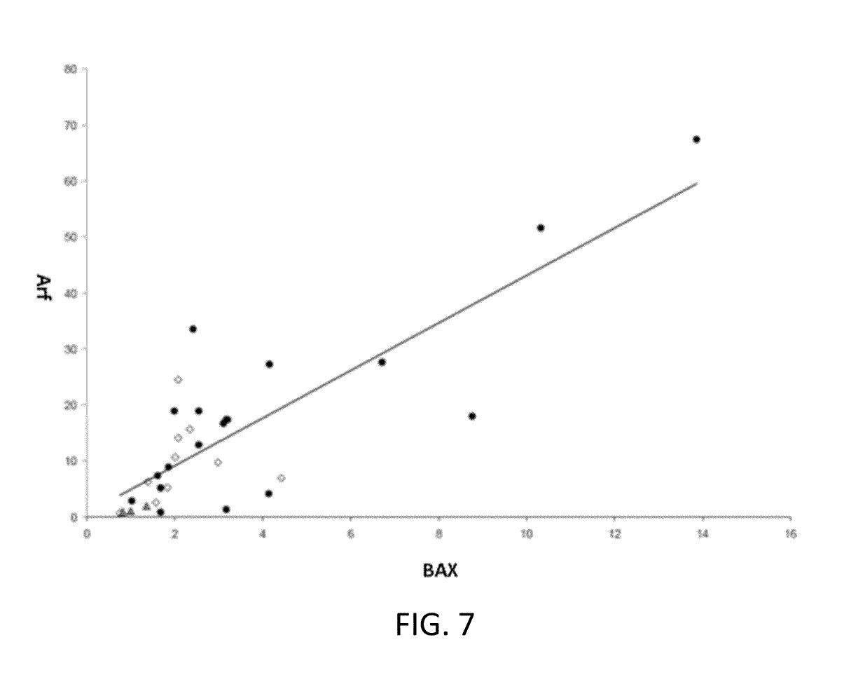

As shown in Example 3 and in FIGS. 6A and 6B and FIG. 7, a highly significant correlation was found herein by the inventors between p19^{Arf} and BAX (apoptotic marker, Bcl-2-associated X protein), respective p21 (senescence marker) and BAX levels, suggesting that in vivo p19^{Arf} does not only induce senescence but simultaneously part of the tumor population becomes committed to apoptosis with both clearly depending on the level of p19^{Arf}. Therefore, in one embodiment the present invention relates to a treatment of uterine fibroids by inducing a cessation of growth and cellular senescence using selected growth factors able to induce Arf expression. Growth factors selected from the group of growth factors comprising FGF-1, bFGF, PDGF-BB, BMP-4 and TGF-beta. In particular, one embodiment of the present invention relates to a growth factor able to induce Arf expression selected from the group comprising FGF-1, bFGF, PDGF-BB, BMP-4 and TGF-beta for treatment of uterine fibroids, wherein a cessation of growth and cellular senescence is induced.

Combinations of growth factors as outlined above for the treatment of uterine fibroids by inducing a cessation of growth and cellular senescence may also be used according to the present invention. Therefore, one embodiment of the present invention relates to a combination of growth factors as defined hereinabove for use in the treatment of uterine fibroids, wherein a cessation of growth and cellular senescence is induced

The present invention relates further to different methods of administration of above-mentioned modulators of the Arf-p53 pathway, i.e. growth factors able to induce Arf expression. It discloses for example, treatment of uterine fibroids by inducing a cessation of growth and cellular senescence by local, intratumoral or systemic administration of selected growth factors as outlined hereinabove. In particular, in one embodiment the present invention relates to growth factors able to induce Arf expression as defined above prepared for treatment of uterine fibroids by a local administration, wherein a cessation of growth and cellular senescence is induced. In another embodiment of the present invention said growth factors are prepared for treatment of uterine fibroids by an intratumoral administration and in a further embodiment said growth factors are prepared for treatment of uterine fibroids by a systemic administration, wherein irrespective of the particular administration form a cessation of growth and cellular senescence is induced.

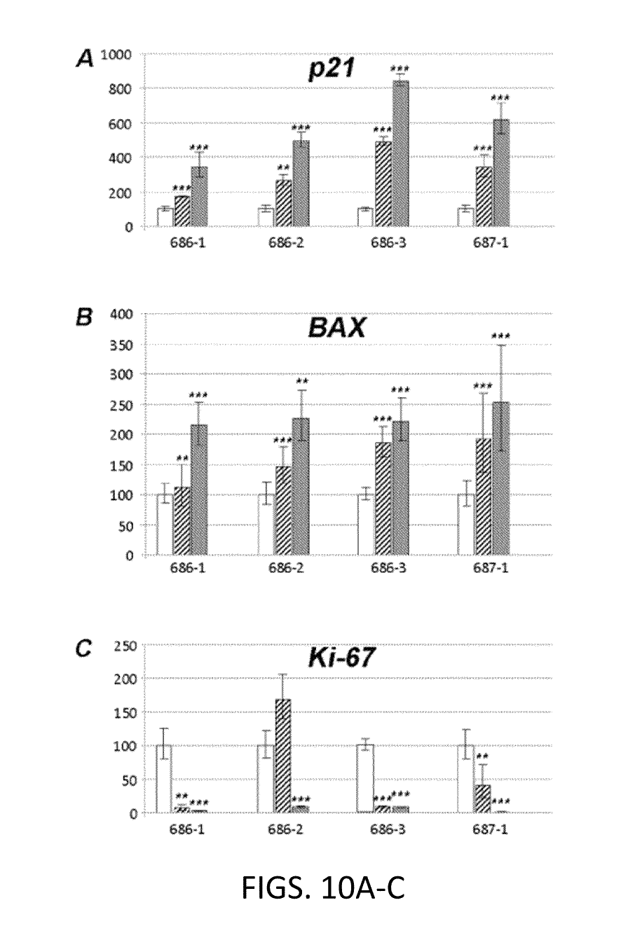

Whatever is the cause of the p14^{Arf} overexpression it activates a p53-MDM2 negative feedback-loop [8] that may govern a delicate balance of the fibroids between proliferative activity and senescence [7]. This makes antagonizing MDM2 an interesting approach towards the growth control of fibroids. Accordingly, nutlin-3 was used, a known MDM2 inhibitor, to antagonize its activity in cell cultures from fibroids. Interestingly, it could be shown that antagonizing MDM2 induces the activity of genes associated with senescence (p21) as well as those associated with apoptosis (BAX) in leiomyoma cells in vitro (see Example 1 and FIGS. 6A and 6B) and in fibroid explants from leiomyomas (Experiment 4 with the corresponding FIGS. 10A-10C).

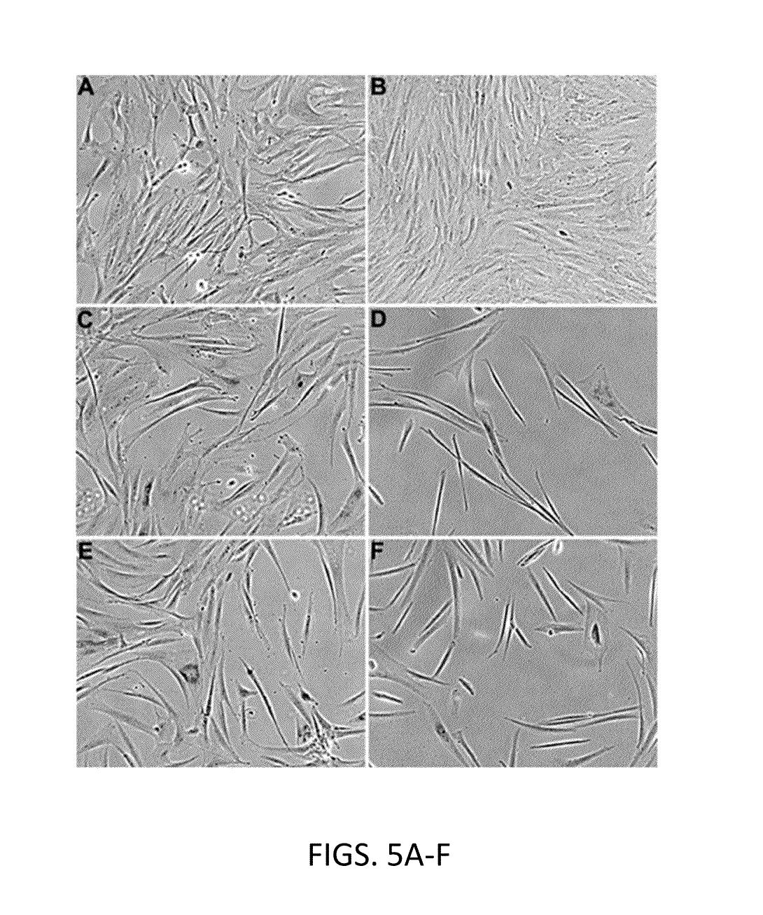

As further shown in Example 2 and FIGS. 5A-5F, 6A and 6B, in vitro administration of nutlin-3, a small-molecule MDM2 antagonist, strongly induced cellular senescence as shown for of β-galactosidase in FIGS. 5A-5F and measured by the percentage of β-galactosidase positive cells as well as by the expression of p21 (FIGS. 6A and 6B). A highly significant correlation between p19Arf and BAX expression in vivo shows that p19Arf triggers apoptosis as well. Thus, it can be concluded that apparently, p19Arf and HMGA2 play a pivotal role in controlling the growth of fibroid cells in vitro. Disturbance of the p53-MDM2 autoregulation by nutlin-3 efficiently induces senescence as well as apoptosis. Moreover, the highly significant correlation between p19Arf and BAX in native fibroids suggests that antagonizing MDM2 offers an opportunity to treat fibroids by simultaneously inducing senescence as well as apoptosis.

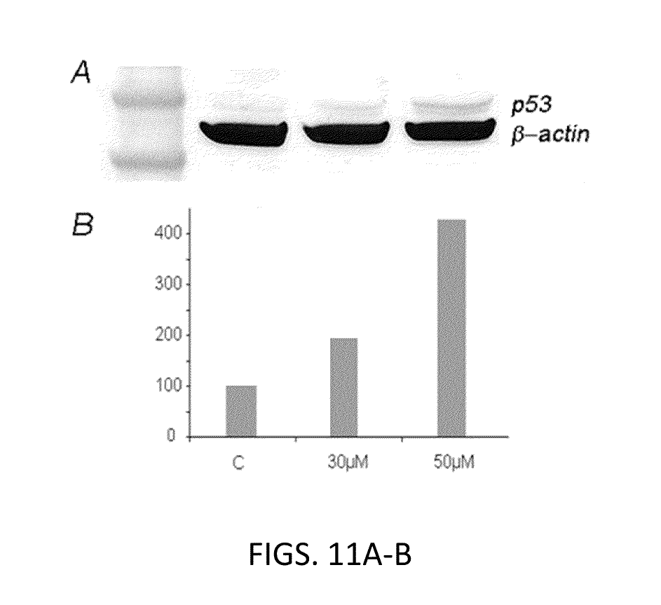

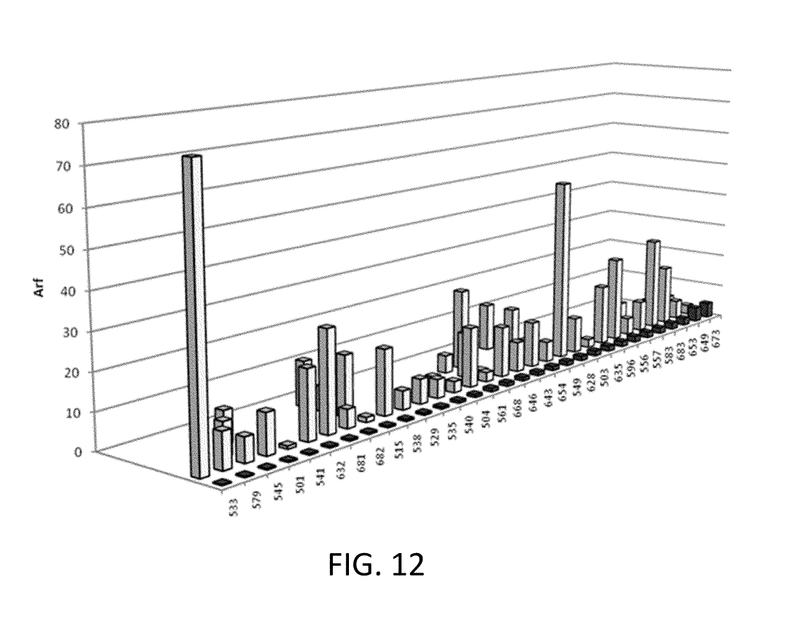

As mentioned above, administration of nutlin-3 strongly induced the expression of p21 (Example 2 and FIGS. 6A and 6B). p21 is thought to be an integral part of the p53-mediated growth-arrest pathway (E1-Deiry et al. 1994). To confirm this dependency, potential changes of p53-levels following nutlin-3 treatment were analyzed by measuring p53 immunohistochemical staining intensity in treated explants tissues and by Western Blot. As shown in Example 4 and in FIGS. 11A and 11B, amounts of p53 increased in the nutlin-3 treated explants in a concentration dependent manner, which could also be confirmed by the observation of an increased number of immunohistochemically stained cells in UL explants tissue compared with matching myometrium; see Table 2. Therefore, the present invention also relates to a treatment of uterine fibroids by inducing a cessation of growth and cellular senescence by administration of modulators of the Arf-p53 pathway as, e.g., MDM2 inhibitors according the aforementioned description. In particular, one embodiment of the present invention relates to modulators of the Arf-p53 pathway prepared for an administration as an MDM2 inhibitor according to the description hereinabove, for treatment of uterine fibroids, wherein a cessation of growth and cellular senescence is induced

Summarizing the aforementioned, a method of treating uterine fibroids, which method comprises administering to a patient in need thereof, an effective amount of an MDM2 inhibitor may also be used according to the present invention. Therefore, in one embodiment the present invention relates to an MDM2 inhibitor for use in the treatment of uterine fibroids

Furthermore, provided herewith is a method of treating uterine fibroids, wherein the patient is administered with an MDM2 inhibitor before undergoing surgery of the uterus. Therefore, in a particular embodiment the present invention relates to an MDM2 inhibitor as described hereinabove, wherein the MDM2 inhibitor is designed to be administered to a patient before undergoing surgery of the uterus.

Uterine fibroids, organized overgrowths of the uterine endometrium such as endometrial polyps and endometriosis (presence of endometrial tissue in locations outside the uterine cavity) may be accompanied by menorrhagia (heavy menstrual bleeding) or dysfunctional bleedings between the periods. Thus, in another aspect, a method for reducing or stopping bleeding in a patient afflicted with uterine fibroids or endometrial polyps or endometriosis which method comprises administering to a patient in need thereof, an effective amount of an MDM2 inhibitor is provided. In a particular embodiment the present invention relates to an MDM2 inhibitor for use in reducing or stopping bleeding in a patient afflicted with uterine fibroids or endometrial polyps or endometriosis

The present invention relates further to different schedules and methods of administration of abovementioned MDM2 inhibitors in the methods of treating uterine fibroids or for reducing or stopping bleeding described supra. Concerning this matter, the method of treating uterine fibroids or for reducing or stopping bleeding as described above, wherein the patient is administered with an oral dosage of an MDM2 inhibitor, preferably wherein the administration is repeated, more preferably wherein the administration is repeated once a year is provided. Therefore, in a one embodiment the present invention relates to the MDM2 inhibitor as defined hereinabove, wherein the MDM2 inhibitor is designed to be administered by an oral dosage, wherein preferably the administration of oral dosage is to be repeated, more preferably wherein the administration of oral dosage is to be repeated once a year.

In the same matter, provided herewith are methods concerning treating uterine fibroids or reducing or stopping bleeding as described above, wherein the patients are administered locally with MDM2 inhibitors using appropriate devices or other types of local administration, preferably wherein the administration is repeated, more preferably wherein the administration is repeated once a year. Therefore, in a further embodiment the present invention relates to the MDM2 inhibitor as described hereinabove, wherein the MDM2 inhibitor is designed to be administered locally using appropriate devices or other types of local administration, preferably wherein the local administration is to be repeated, even more preferably wherein the local administration is to be repeated once a year.

The present invention also relates to a method for treatment of uterine fibroids or a method for reducing or stopping bleeding as described above, wherein the MDM2 inhibitors are selected from the group of inhibitors comprising nutlin-3, nutlins, and derivatives thereof, preferably wherein the administration of MDM2 inhibitors is combined with other medical therapies as e.g. the administration of non-steroidal anti-inflammatory drugs, GnRH (gonadotropin-releasing hormone) agonists, GnRH antagonists, and progesterone-releasing intrauterine devices, preferably wherein further the uterine fibroid of the patient has been diagnosed in accordance with the method to diagnose the growth potential of UL as described hereinabove. Therefore in a further embodiment the present invention relates to an MDM2 inhibitor as defined hereinabove, wherein the MDM2 inhibitor is selected from the group of inhibitors comprising nutlin-3, nutlins, and derivatives thereof, preferably wherein the administration of the MDM2 inhibitor is combined with other medical therapies comprising the administration of non-steroidal anti-inflammatory drugs, GnRH (gonadotropin-releasing hormone) agonists, GnRH antagonists, and progesterone-releasing intrauterine devices, wherein even more preferred the uterine fibroid of the patient has been diagnosed in accordance with the method to diagnose the growth potential of UL as defined hereinabove.

As mentioned above, in a second series of experiments, in addition to tissue from uterine leiomyomas, cells from another tissue of mesenchymal origin, i.e. adipose-tissue derived stem cells (ADSCs) were treated with an MDM2-inhibitor. Mesenchyme tissue is a tissue of mesodermal origin that forms, e.g., connective tissue, blood, smooth muscles and together with the coelomic epithelium the uterus (Brenner and Maslar, 1988; Langman, 1979). As any adult tissue with repair and/or regenerative capabilities, mesenchymal tissues harbor tissue-specific stem cells with the ability of repair and/or reconstitute a specific tissue. A number of cell types come from mesenchymal stem cells, including cartilage, bone, muscle, adipose tissue and the two layers of the adult uterus: the endometrial stroma and the myometrial muscle layers (Kurita et al., 2001; Teixeira et al., 2008; Schipani and Kronenbert, 2009; Cook and Cowan, 2009). Comparable to the effect on UL cells, said treatment led to an decreased self-renewal capacity of these cells as well, pointing at a general usefulness of the methods and compounds of the present invention in the treatment of diseases characterized by an aberrant growth of mesenchymal stem cells and their descendants.

Therefore, in another aspect the present invention relates to an MDM2 inhibitor for use in reducing the number of mesenchymal stem cells and/or to abrogate their self-renewal capacity.

Herein, in one embodiment the present invention relates to an MDM2 inhibitor for use in the treatment of diseases characterized by an aberrant growth of mesenchymal stem cells and their descendants, preferably wherein the diseases are selected from the group comprising endometriosis, adenomyosis, endometrial hyperplasia, leiomyoma, lipoma, hamartoma of the lung, fibroadenoma of the breast, adenoma of the salivary gland, and aggressive angiomyxomas.

In a further embodiment the present invention relates to the MDM2 inhibitor for use in the treatment of diseases characterized by an aberrant growth of mesenchymal stem cells and their descendants, wherein the diseases are selected from the group of diseases characterized by recurrent clonal chromosome aberrations involving chromosomal bands 12q14˜15 or 6p21, preferably wherein the diseases are selected from the group of diseases showing recurrent clonal chromosomal alterations targeting the gene loci of either of one of the genes encoding high mobility AT-hook proteins HMGA1 or HMGA2.

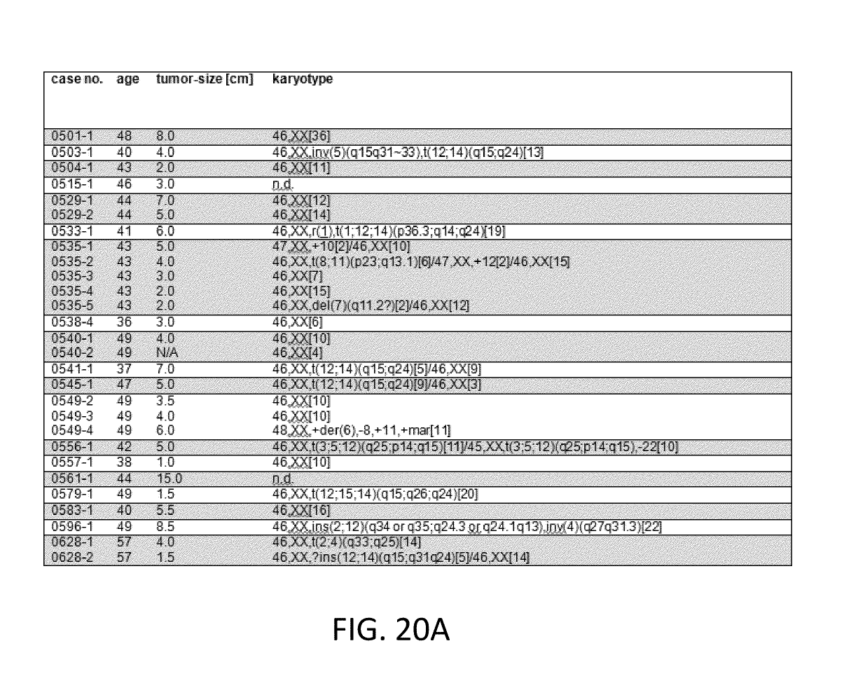

Furthermore, obesity, as a disorder characterized by an overgrowth of the adipose tissue may be treated according to the methods of the present invention as well, as shown in Example 8 and FIGS. 19A and 19B by treatment of ADSCs with the MDM2-inhibitor nutlin-3. Thus, in another aspect the present invention relates to an MDM2 inhibitor for use in the treatment of obesity.

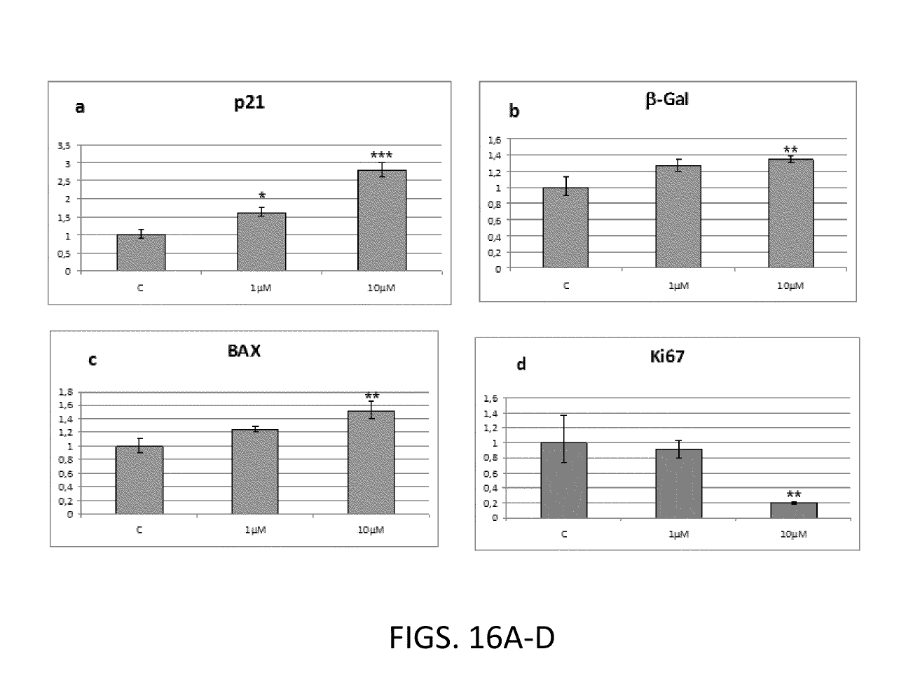

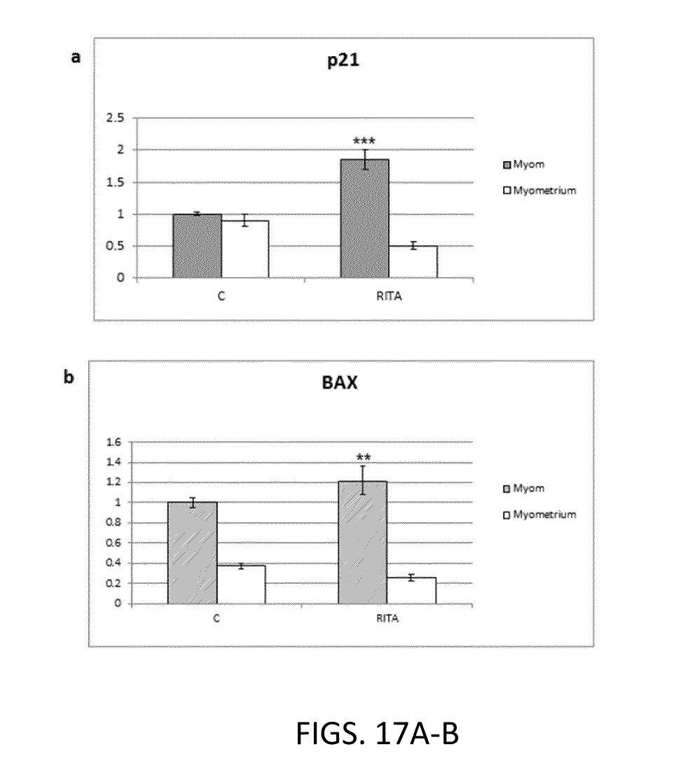

According to the present invention, the MDM2 inhibition may be achieved using several methods. As shown in Example 5, treatment of the cells by siRNAs specifically designed against MDM2 lead to an increased expression of p21 (FIG. 15A) and BAX (FIG. 15B) and to a decreased expression of Ki-67 (FIG. 15C) indicating that such treatment can reduce the growth potential of cells from uterine leiomyomas and at the same time increase the expression of two genes linked to senescence and apoptosis, respectively. As further shown in Example 6 and Example 7 and FIGS. 16A-D, 17A-B, and 18A-B, generally all other MDM2 inhibitors, such as appropriate RNA molecules, and small molecules as, e.g., RITA and tenovin-1 may be used analogous to nutlin-3 in a way to induce senescence and apoptosis in aberrantly growing mesenchymal stem cells and their descendants, e.g., leiomyoma cells. Therefore, in one embodiment the present invention relates to the MDM2 inhibitor for use in reducing the number of mesenchymal stem cells and/or to abrogate their self-renewal capacity or for use in the treatment of diseases characterized by an aberrant growth of mesenchymal stem cells and their descendants, wherein the MDM2 inhibitor is selected from the group of molecules comprising small molecules, antibodies, siRNA, microRNA, aptamers, and spiegelmers, preferably wherein the small molecules are selected from or having the same structure as nutlins, tenovins, tenovin-1, tenovin-6, or RITA.

Expression of genes or levels of specific proteins in cells or organs can be reduced by techniques using antisense molecules, for example. “Antisense molecules” or “antisense reagents” can, in the present context, be any molecule that hybridizes by a sequence specific base pairing to a complementary DNA and/or RNA sequence. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid nonspecific binding of the antisense compound to non-target sequences under conditions in which specific binding is desired, i.e., under physiological conditions in the case of in vivo assays, and in the case of in vitro assays, under conditions in which the assays are performed. Typical “antisense molecules” or “antisense reagents” are any oligonucleotide, such as DNA, RNA, any peptide nucleic acid, any other nucleic acid derivative, or mimic and/or derivative thereof. The target sequence is not restricted to the “sense” or “coding” strand of mRNA, although this is often the target. According to the present invention “antisense molecules,” or “antisense constructs” can be employed which are used interchangeably in the present text. In one embodiment of the present invention the use of oligonucleotides, for use in modulating the function of nucleic acid molecules encoding genes, in particular of the MDM2 or the HMGA2 gene is addressed. This is accomplished by providing antisense compounds which specifically hybridize with one or more nucleic acids encoding a target gene, such as the MDM2 or the HMGA2 gene.

As used herein, the term “target nucleic acid” encompasses a DNA encoding said gene, and/or an RNA (including pre-mRNA and mRNA) transcribed from such DNA. The specific hybridization of an oligomeric compound with its target nucleic acid interferes with the normal function of the nucleic acid. This modulation of function of a target nucleic acid by compounds which specifically hybridize to it is generally referred to as “antisense” (when the target is RNA) or “antigene” (when the target is DNA). The functions of DNA to be interfered with include replication and transcription. This effect is referred to as “antigene”. Such interactions may occure by binding of the “antigene” molecule to the DNA double-helix as a third strand in its major groove forming a structure also known as “triplex DNA” or “triple helix DNA” (Frank-Kamenetskii, Annu. Rev. of Biochem. 64 (1995), 65-95; Rusting et al., Nucleic Acids Res. 33 (2005), 3025-3032). The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA and is referred to as “antisense”. However, the distinction between “antisense” and “antigene” is not absolute.

The overall effect of such interferences with target nucleic acid function is a specific modulation of the expression of said essential gene. In the context of the present invention, “modulation” means either an increase (stimulation) or a decrease (inhibition) in the expression of a gene. In the context of the present invention, in particular concerning modulation of MDM2, inhibition is the preferred form of modulation of gene expression.

In the present invention, antisense molecules can be selected from the group consisting of oligonucleotides, oligonucleotide analogues, oligonucleotide mimics, such as for example PNA, locked nucleic acids (LNA), phosphorothioate, 2′-methoxy-, 2′-methoxyethoxy-, morpholino, phosphoramidate oligonucleotides or the like. In the present invention, antigene molecules can furthermore be selected from the group consisting of triplex forming or strand invading oligonucleotides, oligonucleotide analogues, oligonucleotide mimics, such as for example PNA, locked nucleic acids (LNA), phosphorothioate, 2′-methoxy-, 2′-methoxyetyhoxy-, morpholino, phosphoramidate oligonucleotides or DNA minor groove binding polyamides (oligo pyrroles/imidazoles etc.) as described (Gottesfeld et al., Gene Expr. 9 (2000), 77-91; Dervan and Bürli, Curr. Opin. Chem. Biol. 3 (1999), 688-693) or the like.

The term “oligonucleotide(s)” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally occurring nucleobases, sugars and covalent internucleoside (backbone) linkages which function similarly or combinations thereof. Such modified or substituted oligonucleotides are often preferred over native forms because of desirable properties such as, for example, enhanced cellular uptake, enhanced affinity for nucleic acid target and increased stability in the presence of nucleases and other enzymes, and are in the present context described by the terms “oligonucleotide analogues” or “oligonucleotide mimics”.

The antisense compounds in accordance with this invention preferably comprise from 7 to 80 nucleobase units, preferably not more than 30 nucleobase units to avoid an interferon response (Manche et al., Mol. Cell. Biol. 12 (1992), 5238-5248). The term “nucleobase units” is used in the present text to describe both the number of nucleotides in an oligonucleotide and the number of nucleobase-carrying monomers of an oligonucleotide mimetic. Particularly preferred antisense compounds are antisense oligonucleotides, even more preferably those comprising from 14 to 29 nucleobases. Most preferred are short RNA based antisense oligonucleotides comprising around 20 nucleobases, i.e. from 18 to 26 nucleobases, of two particular molecular classes, either single stranded (miRNA) or double stranded (siRNA).

Unmodified, naked antisense molecules were reported to be internalized poorly by cells, whether or not they are negatively charged (Grey et al., Biochem. Pharmacol. 53 (1997). 1465-1476, Stein et al., Biochemistry 32 (1993), 4855-4861. Bennet et al., Mol. Pharmacol. 41 (1992), 1023-1033). Therefore, the oligonucleotides may be modified or used in compositions with other agents such as lipid carriers (Fattal et al., Adv. Drug Deliv. Rev. 56 (2004), 931-946), microparticles (Khan et al., J. Drug Target 12 (2004), 393-404) or by covalent conjugation to cell-penetrating peptides (CPP) allowing translocation of the antisense molecules through the cell membrane; see Lysik and Wu-Pong, J. Pharm. Sci. 92 (2003), 1559-1573 for an review.

As used herein, the term “targeting molecule” refers to any agent (e.g., peptide, protein, nucleic acid polymer, aptamer, spiegelmer or small molecule) that specifically binds to a target of interest. The target of interest may be a tissue, a cell type, a cellular structure (e.g., an organelle), a protein, a peptide, a polysaccharide, or a nucleic acid polymer. In some embodiments, the targeting molecule is any agent that specifically binds to one or more neurons or nerves of a subject. As used herein, the term “aptamer” refers to a DNA or RNA molecule that has been selected from random pools based on their ability to bind other molecules with high affinity specificity based on non-Watson and Crick interactions with the target molecule (see, e.g., Cox and Ellington, Bioorg. Med. Chem. 9 (2001), 2525-2531; Lee et al., Nuc. Acids Res. 32 (2004), D95-D100). Aptamers can be selected which bind nucleic acid, proteins, small organic compounds, vitamins, inorganic compounds, cells, and even entire organisms.

The peptides and aptamers of the present invention are synthesized by any suitable method. For example, targeting peptides and aptamers of the present invention can be chemically synthesized by solid phase peptide synthesis. Techniques for solid phase synthesis are described, for example, by Barany and Merrifield (1979) Solid-Phase Peptide Synthesis; pp. 1-284 in The Peptides: Analysis, Synthesis, Biology, (Gross. and Meinehofer, eds.), Academic, New York, Vol. 2, Special Methods in Peptide Synthesis, Part A.; Merrifield, J. Am. Chem. Soc, 85 (1963), 2149-2154; and Stewart and Young (1984) Solid Phase Peptide Synthesis, 2nd ed. Pierce Chem. Co., Rockford, Ill.

Spiegelmers are nucleic acids comprising a number of L-nucleotides which show binding activities towards a target or a part thereof. The basic method of Spiegelmer generation is subject to the international patent application WO 1998/008856 the disclosure of which is incorporated herein by reference. Basically, this method relies on the so-called SELEX technique as described, e.g., in U.S. Pat. No. 5,475,096. The method uses combinatorial DNA or RNA libraries comprising a randomised stretch of about 10 to about 100 nucleotides which are flanked by two primer binding regions at the 5′ and 3′ end. The generation of such combinatorial libraries is, for example, described in Conrad et al., Methods Enzymol., 267 (1996), 336-367. Such a chemically synthesized single-stranded DNA library may be transferred into a double-stranded library via polymerase chain reaction.

Such a library may already be used for selection purpose. The selection occurs such that the, typically single-stranded, library is contacted with a target molecule and the binding elements of the library are then amplified. By repeating these steps several times oligonucleotide molecules may be generated having a significant binding activity towards the target used.

Spiegelmers, as said above, are actually L-polynucleotides which are generated such that D-polynucleotides are selected against a target molecule which is present in its non-naturally occurring enantiomer, and the nucleic acid binding thereto is then synthesized using L-nucleotides creating the L-polynucleotide, which is the Spiegelmer. This L-polynucleotide is capable of binding to the target molecule in its naturally occurring form. In case the target is a protein or peptide the non-naturally occurring enantiomer is the D-protein/peptide and the naturally occurring enantiomer is the L-protein/peptide.

Targets against which the Spiegelmers can actually be generated are viruses, proteins, peptides, nucleic acids, small molecules like metabolites of the metabolic pathways, pharmaceutically active compounds and their metabolites.

In connection with the present invention this specificity of the Spiegelmers may actually be used to target a certain structure, compound, process, condition or disease. This may be done such that the structure targeted or aimed at in connection with the in vivo imaging process comprises a chemical compound which is used as the target in the Spiegelmer generation process. As the in vivo imaging may also target a certain process condition or disease, an appropriate approach to the use of Spiegelmers in in vivo imaging methods aiming at these, would be to generate Spiegelmers against a chemical compound being involved in said process, condition or disease.

In some embodiments, the targeting molecule further comprises a cargo. In some embodiments, the peptide or aptamer is directly bound to a cargo. In some embodiments, the peptide or aptamer is indirectly (e.g., via a linker) bound to a cargo. In some embodiments, two or more peptides or aptamers are directly or indirectly bound to a cargo. In some embodiments, the cargo is a drug. In some embodiments, the cargo is a fluorescent moiety.

In some embodiments, the targeting molecule further comprises a drug. All drugs that act on a neuron or nerve (or a component thereof) are encompassed within the term “drug.” Specific examples of drag given herein are illustrative and are not meant to limit the drugs for use with the targeting molecules disclosed herein.

In some embodiments, the peptide or aptamer is directly bound to a drug. In some embodiments, the peptide or aptamer is indirectly (e.g., via a linker) bound to a drug. In some embodiments, two or more peptides or aptamers are directly or indirectly bound to a drug.

The examples which follow further illustrate the invention, but should not be construed as to limit the scope of the invention in any way. Detailed descriptions of conventional methods, such as those employed herein can be found in the cited literature; see also “The Merck Manual of Diagnosis and Therapy” Seventeenth Ed. ed by Beers and Berkow (Merck & Co., Inc. 2003). The practice of the present invention will employ, unless otherwise indicated, conventional techniques of cell biology, cell culture, molecular biology, transgenic biology, microbiology, recombinant DNA, and immunology, which are within the skill of the art.

Suitable regimens for therapeutic administration and methods for preparing pharmaceutical compositions of the invention are within the skill in the art, for example as described in Remington's Pharmaceutical Science, 17th ed., Mack Publishing Company, Easton, Pa. (1985) and update version Remington: The Science and Practice of Pharmacy (2000) by the University of Sciences in Philadelphia, ISBN 0-683-306472, the entire disclosure of both documents which is incorporated herein by reference.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107.

### EXAMPLES

### Material and Methods

### Tissue Samples and Cells

Samples of uterine leiomyomas and myometrium were taken during surgery, immediately frozen in liquid nitrogen, and stored at −80° C. for RNA isolation. As a reference for the expression of the p16^{Ink4a}/p19^{Arf} locus human adipose tissue derived stromal cells (ADSCs) isolated from subcutaneous adipose tissue were used. Subcutaneous adipose tissue was taken during surgery from patients admitted to the Department of General and Vascular Surgery, Clinical Center Bremen-Nord, Bremen, Germany. For cell culture the tissue was transferred into sterile Hank's solution and minced into small pieces followed by a treatment with 0.26% (200 U/ml) collagenase (Serva, Heidelberg, Germany). After 1-2 hours, the dissociated cells were transferred into sterile 25 cm² cell culture flasks containing 5 ml medium 199 supplemented with 20% fetal bovine serum (FCS) (Invitrogen, Karlsruhe, Germany) and antibiotics (2% penicillin-streptomycin) (Biochrom, Berlin, Germany). The cultures were incubated in 5% CO₂ air at 37° C. and medium was changed every 2-3 days. Cultures were passaged when reaching 80% confluence using 1× concentrated TrypLE Express in a PBS-EDTA buffer. The cells were subcultured in medium 199 with 10% FCS and antibiotics. Subcultivation was performed until passages 14 (cells of the UL with t(2;12)), and 13 (UL with normal karyotype). Total RNA of every passage was extracted 72 h after medium change.

### Treatment by Nutlin-3

For treatment by nutlin-3 cells of an UL with an apparently normal karyotype (5th in vitro passage) were plated in Leighton-tubes at a density of 200,000 on 10 mm×50 mm cover slips 24 h before incubation with nutlin-3 (Biomol, Hamburg, Germany). Cells of the UL were treated with 30 μM and 50 μM nutlin-3 for 24 h and for 72 h, respectively. As controls cells were cultured in medium 199 supplemented with 20% FCS without nutlin-3 for 24 h and 72 h.

### In Situ β-Galactosidase Staining and Mitotic Rate

After incubation with nutlin-3 β-galactosidase staining of senescent cells was performed using a commercially available in situ β-galactosidase staining kit (Agilent, Waldbronn, Germany) according to the manufacturer's instructions. After incubation with staining solution at 37° C. for 24 h cells were washed two times with 1×PBS and analyzed using an axioscope (Zeiss, Gottingen, Germany). The numbers of β-galactosidase-positive cells and of mitotic cells were counted using photomicrographs of ten randomly selected fields at 10× magnification.

### RNA Isolation

Total RNA was isolated from frozen tissue samples and from cell culture by using the miRNeasy Mini Kit (Qiagen, Hilden, Germany) and DNase I digestion was performed following the manufacturer's instructions.

cDNA-Synthesis

250 ng of total RNA were reverse transcribed with M-MLV reverse transcriptase (Invitrogen, Karlsruhe, Germany), RNase Out (Invitrogen, Karlsruhe, Germany), random hexamers and dNTPs according to the manufacturer's instructions. RNA was denatured at 65° C. for 5 min and subsequently kept on ice for 1 min. After adding the enzyme to the RNA primer mixes, samples were incubated for 10 min at 25° C. to allow annealing of the random hexamers. Reverse transcription was performed at 37° C. for 50 min followed by inactivation of the reverse transcriptase at 70° C. for 15 min.

### Quantitative Real-Time PCR

Relative quantification of transcription levels was carried out by real-time PCR analyses using the Applied Biosystems 7300 Real-Time PCR system (Applied Biosystems, Darmstadt, Germany). Commercially available gene expression assays (Applied Biosystems, Darmstadt, Germany) were used for quantification of mRNA of human HMGA2 (Hs00171569 ml), p19Arf (Hs00924091), BAX (Hs00180269_m1), MDM2 (Hs01066930_m1), p21 (Hs99999142), Ki-67 (Hs00606991_m1), p16^{Ink4a} (Hs00923893_m1) and β-galactosidase (Hs01035162). The housekeeping gene hypoxanthine phosphoribosyltransferase (HPRT) served as endogenous control with the primers 5′-GGC AGT ATA ATC CAA AGA TGG TCA A-3′ (forward) (SEQ ID NO.:1), 5′-GTC TGG CTT ATA TCC AAC ACT TCG T-3′ (reverse) (SEQ ID NO.:2), and the fluorescent probe 5′-6-FAM-CAA GCT TGC TGG TGA AAA GGA CCC C-TAMRA-3′ (SEQ ID NO.:3); see also Specht et al. (2001). All qRT-PCR experiments were done in triplicate.

### Cytogenetic and Molecular-Cytogenetic Studies

Chromosome analyses and fluorescence in situ hybridization were performed following routine techniques as described previously (Klemke et al. 2009).

### Statistical Analysis

The statistical significance of differences was assessed by the student's t test. In all comparisons, p<0.05 was considered being statistically significant and p<0.01 was considered being highly significant.

### Example 1

### In Vitro Senescence Accompanied by a Decrease of the Expression of HMGA2 and an Increase of p19Arf

22 uterine leiomyomas and seven myometrial tissues have been tested by qRT-PCR for their relative expression of p16^{Ink4a} and p19^{Arf} mRNA. Based on cytogenetic analyses, the leiomyomas were further divided into a group with 12q14˜15 aberrations (n=10) and another group with other karyotypes (n=12). While in the control (adipose tissue derived stromal cells, ADSCs, 6^{th} in vitro passage) but in none of the samples an expression of p16^{Ink4a} mRNA was detectable, p19^{Arf} mRNA was detectable in all samples measured and was found in the range detected in ADSCs (FIG. 1). Generally, the expression of p19^{Arf} in leiomyomas varied over a broad range while it was almost identical in the myometrial tissue. The relative p19^{Arf} mRNA expression in the leiomyomas significantly (p<0.05) exceeded that in myometrial tissue with an average p19^{Arf} mRNA-level of 0.062 (range: 0.009-0.104) in myometrial tissue and of 0.704 (range: 0.034-3.675) in uterine leiomyomas by more than 10-fold. Furthermore, in leiomyomas with 12q14˜15 aberrations p19^{Arf} mRNA was expressed at higher levels than in UL with other karyotypes (1.073 vs. 0.396, p<0.05). Comparing the p19^{Arf} expression of UL with 12q14˜15 aberrations with myometrial tissue resulted a 17.4-fold (p<0.01) higher expression in the aberrant leiomyomas. Because in neural stem cells a repression of the Ink4a/Arf locus by HMGA2 has been described (Nishino et al., 2008) it was tested next whether a correlation between the expression level of HMGA2 mRNA and p19^{Arf} mRNA exists. As a rule, leiomyomas with 12q14˜15 rearrangements are known to overexpress HMGA2 (Gross et al., 2003; Klemke et al., 2009) and also in this series its expression exceeded that of the fibroids with other cytogenetic subgroups in nearly all cases. Nevertheless, the expression between individual tumors of that group varied strongly. The analysis did not reveal a simple linear relationship between the expression of HMGA2 and p19^{Arf} (FIG. 2). Rather, HMGA2 may stimulate Arf at an intermediate level of overexpression with a decreasing stimulation observed in tumors strongly overexpressing HMGA2. Because a positive feedback loop between TP53 and MDM2 is well documented, it was analyzed whether enhanced expression of p19Arf triggers an increased expression of MDM2 as well. A highly significant linear correlation (P<0.001) between the expressions of p19Arf and MDM2 was noted (FIG. 4). Moreover, fibroids with 12q14_{—}15 rearrangements expressed significantly (P<0.05) higher levels of MDM2 mRNA than those with an apparently normal karyotype.

### Example 2

### Antagonizing MDM2 Induces Senescence as Well as Apoptosis in Fibroid Cells In Vitro

These findings obtained in accordance with the present invention suggest that in vitro senescence of leiomyoma cells is controlled by the HMGA2-p19Arf axis. Thus, a similar mechanism seems to account for the in vivo aging of stem cell populations as well as for in vitro senescence of cells from uterine fibroids. Thus, it is tempting to assume that in vivo senescence contributes to the growth control of UL as well. There are some recent data supporting this idea. It was demonstrated, e.g., that uterine leiomyomas express significantly higher levels of p19Arf than myometrial tissue and that in leiomyomas delicate balances along the HMGA2-p19Arf-MDM2-p53-p21 axis seem to exist that dictate the fate of the individual fibroids. Because of the possible similarities between in vitro and in vivo senescence of these tumors it was addressed next whether in vitro this balance can be disturbed by MDM2 inhibitors. A class of well known small molecule inhibitors of MDM2 are nutlins (Brown et al., Nat. Rev. Cancer 9 (2009), 862-873), small molecules that inhibit the interaction of MDM2 with p53 (Vassilev et al., Science 303 (2004), 844-848.). Accordingly, nutlin-3 has been used to inhibit MDM2 in cell cultures from two UL. To determine whether this treatment can induce senescence of the cultures, the percentage of β-galactosidase positive tumor cells in controls and after treatment with nutlin-3 (30 and 50 μM) for two different times (24 h, 72 h) was determined Compared to each of the controls, the number of positive cells clearly increased after treatment (FIGS. 5A-F, 6A and 6B) indicating that the cells are sensitive to an inhibition of MDM2. Accordingly, mitoses were nearly absent in any of the cultures treated by nutlin-3. To examine whether the inhibition of MDM2 only results in an increase of senescence but not of apoptosis it was examined whether the treatment with nutlin-3 in vitro results only in an increase of p21 but not of the apoptotic marker BAX. Normal fibroblasts are known to be resistant against nutlin-induced apoptosis (Efeyan et al., Cancer Res. 67 (2007), 7350-7357).

However, contrary to these expectations cells harvested for qRT-PCR analysis of p53 targets, i.e. p21, BAX as well as of the expression of β-galactosidase have shown that the treatment of cells with nutlin-3 clearly increased the expression of p21, β-galactosidase, and BAX whereas it reduced that of Ki-67.

### Example 3

### p19Arf Drives Senescence as Well as Apoptosis in Uterine Fibroids

Because data showed that inhibition of MDM2 triggers the senescence pathway as well as apoptosis the inventors were interested whether p19Arf/MDM2 may also correlate with the expression of an apoptosis marker, i.e. BAX in vivo. Thus, samples from a total of 29 UL of both most frequent karyotypic groups were analyzed for possible correlations between p19Arf and BAX and p21 and BAX. Interestingly, a highly significant correlation was found in both analyses (FIG. 7, 8) suggesting that in vivo p19Arf does not only induce senescence but simultaneously part of the tumor cell population becomes committed to apoptosis with both clearly depending on the level of p19Arf. This latter finding is confirmed by a highly significant correlation between the expression of p21 and BAX.

### Discussion

Uterine fibroids are highly common benign tumors of women in their reproductive age. In numerous studies, the influence of the hormonal environment as well as, e.g., gene polymorphisms, epigenetics, a familial disposition, or even oncogenic viruses (Romagnolo et al., 1996; Webster et al., 1998; Bullerdiek, 1999; Asada et al., 2008) and recently the de-regulation of micro-RNA genes (Luo and Chegini, 2008; Marsh et al., 2008; Wei and Soteropoulos, 2008) have been considered as factors related to the development of these frequent tumors clonally arising from the proliferation of smooth muscle cells (SMC). Nevertheless, although cytogenetic analyses allow distinguishing between different cytogenetic subtypes of uterine leiomyomas and point to genes of pathogenetic relevance for the disease, generally very little is known about downstream mechanisms linked to the pathogenesis of the disease. In one of these subtypes rearrangements of the chromosomal region 12q14˜15 lead to an enormous albeit varying upregulation of HMGA2. Recent data suggesting a relationship between the expression of HMGA2 and the repression of the senescence-associated proteins encoded by the p16^{Ink4a}/p19^{Arf} locus prompted the inventors to examine whether HMGA2 exerts at least in part its stimulation of SMC growth seen in fibroids by repression of their senescence program.

In contrast to what had been expected, uterine fibroids generally expressed one gene of the Cdkn2a locus, i.e. Arf, significantly higher than apparently normal myometrium. In contrast, no such differences were noted for Ink4a, the other gene of the locus. The two proteins encoded by the Cdkn2a locus, i.e. p16^{Ink4a} and p19^{Arf}, are cell cycle inhibitors that both have been linked to cellular senescence. Of these p16 blocks phosphorylation and inactivation of the retinoblastoma protein (pRb) whereas p19 blocks via its interaction with MDM2 the ubiquitylation and degradation of p53. In primary fibroblasts, p19^{Arf} seems to mediate a network that enforces Ras-induced cell cycle arrest and tumor suppression (Sebastian and Johnson, 2009) and generally upregulation of p19^{Arf} is part of the so-called oncogene induced senescence (OIS), a term coined to indicate a form of senescence resulting from activated oncogenes and oncogenic viruses, respectively. Besides being part of the p53 network, p19^{Arf} is also known to have p53-independent anti-proliferative activities. Mice lacking Arf, Mdm2, and p53 in combination develop a much broader spectrum of tumors than animals lacking Arf or p53 alone. The tumors arise rapidly and can appear simultaneously at independent sites, where they can involve mesenchymal, epithelial, hematopoietic, or neural cells suggesting that the Arf-Mdm2-p53 pathway is not strictly linear (Lowe and Sherr, 2003). In line with these findings Arf-null mice become blind soon after birth, a phenotype not seen in mice lacking p53 (McKeller et al., 2002).

Generally, both proteins of the Cdkn2a locus have been implicated in the suppression of neoplastic growth and the Cdkn2a locus is among the most frequently inactivated gene loci in human cancers (Lowe and Sherr, 2003). Mice carrying a targeted deletion of the Ink4a locus that eliminates both p16^{Ink4a} and p19^{Arf} develop spontaneous tumors at an early age and are highly sensitive to carcinogenic treatments (Serrano et al., 1996). In line with these findings, it has recently been shown that cells with low endogenous p19^{Arf} levels and immortal fibroblasts deficient in components of the Arf-Trp53 pathway yield induced pluripotent stem cell colonies with up to threefold faster kinetics and at a significantly higher efficiency than wild-type cells (Utikal et al., 2009).

As to the development of leiomyomas, the higher expression of the senescence-associated p19^{Arf} may account for the lower inherent growth potential which, contrary to expectations, has been observed in independent studies comparing the growth dynamics of ex vivo cultures of normal myometrial cells and of leiomyoma cells (Carney et al., 2002; Loy et al., 2005; Chang et al. 2009). Simultaneously, in a quite recent paper Chang et al. (2009) were also able to demonstrate that leiomyomas exhibited fewer stem and progenitor cell characteristics, respectively, than matching myometrium. Generally, the presence of stem cell-like population in the myometrium has been postulated and, e.g., been linked to changes of uterine smooth muscle tissue during post partum involution (Shynlova et al., 2009). Accordingly, a study by Ono et al. (2007) revealed the existence of a stem cell-like side population of quiescent multipotent human myometrial cells which they referred to as myoSP cells. Based on these findings the authors of said study have suggested that repeated menstruation-induced hypoxia may cause clonal proliferation of a myoSP cell that would ultimately result in the development of a leiomyoma which as a second event can acquire cytogenetic abnormalities. In line with this hypothesis, tracing back the origin of uterine leiomyomas to initially multipotent stem cells is the two or more lineage differentiation that is rarely seen in UL and, e.g., gives rise to lipoleiomyoma or chondroleiomyoma.

However, the highly significant upregulation of p19^{Arf} in UL offers prima facie supporting data explaining their lower in vitro growth potential compared to normal tissue and does neither exclude a stem cell-origin of UL nor contradict the loss of stem cell characteristics. In addition, the fact that fewer stem cell colonies are formed from tissue taken from UL than from myometrium (Chang et al., 2009) may be due to advanced senescence simultaneously reflected by the higher expression of p19^{Arf}. Nevertheless, a slightly more complicated model is preferred within the present invention that fits with the upregulation of HMGA2 and the recent data on its function as well:

The cell of UL origin may be a myoSP cell or a more differentiated cell where an unknown oncogenic event is supposed to trigger a mild form of OIS leading to an upregulation of p19^{Arf} compatible with slow clonal proliferation. In the subset of UL with chromosomal rearrangements of 12q14˜15 the drastic upregulation of HMGA2 has a two-fold effect. First, it behaves like an activated oncogene inducing OIS in a dose dependent manner. Secondly, having reached a certain concentration it dominantly acts by repressing p19^{Arf} akin to its function in stem cells. Finally, the interplay between HMGA2 and p19^{Arf} which apparently clearly depends on the degree of overexpression of HMGA2 may be one of the factors determining the final size of UL.

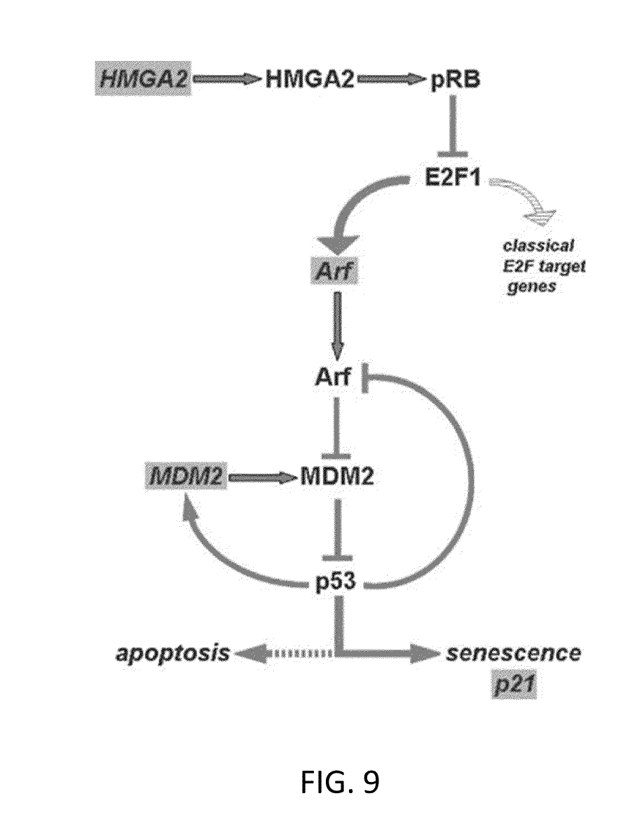

In FIG. 9, available data on the relevant pathway are summarized. The cell of UL origin may be a myoSP cell or a more differentiated cell where an unknown oncogenic event is supposed to trigger a mild form of OIS leading to an upregulation of p19Arf compatible with slow clonal proliferation. In the subset of UL with chromosomal rearrangements of 12q14˜15, the drastically upregulated HMGA2 expression can induce or strengthen the OIS in a dose-dependent manner. Secondly, the expression of p19Arf and the corresponding pathway increases with the growth of the tumor cell population as reflected by the correlation between p19Arf and CDKN1A mRNA with tumor size. Overall, the induction of this pathway as well as of the positive feedback loop involving MDM2 may be a reminiscence of the stem cell origin of uterine fibroids and may help the tumor cells to maintain their genomic integrity despite high levels of HMGA2. Finally, the interplay between HMGA2 and p19Arf/CDKN1A, which apparently, among other factors, depends on the degree of overexpression of HMGA2, seems to be one of the key elements determining the final size of UL. Interestingly, OIS has been considered recently also as a cause for the spontaneous cessation of growth of pituitary adenomas (Mooi, 2009), benign endocrine tumors that share with leiomyomas their high prevalence. In the adult population a prevalence of pituitary microadenomas of up to 25% has been described.

In summary, OIS seems to be an important phenomenon in the development of UL and cessation of their growth. HMGA2, encoded by a gene targeted in a frequent genetic subtype of UL, and the senescence-associated p19^{Arf} are assumed to be major players interacting in different ways to dictate the fate of an individual fibroid.

Moreover, the positive feedback between p53 and MDM2 as revealed by gene expression studies showing a strongly positive correlation between the expression of MDM2 and p19Arf is expected to have therapeutic implication as well: As shown herein, senescence as well as apoptosis of UL cells in vitro can be induced by antagonizing MDM2, a negative regulator of p53 that modulates its transcriptional activity and stability (Carvajal et al., Cancer Res. 65 (2005), 1918-1924; Efeyan et al., Cancer Res. 67 (2007), 7350-7357; Kumamoto et al., Cancer Res 68 (2008), 3193-3203; Secchiero et al., Circ Res 100 (2007), 61-69). In cancer, the use of MDM2 antagonists as effective therapeutics is limited by the requirement not only of wild-type p53 but also of functional signaling in the p53 pathway (Tovar et al., Proc. Natl. Acad. Sci. USA 103 (2006), 1888-1893). In view of the results shown herein, it is prudent to assume usefulness of MDM2 antagonists in the therapy of a common type of benign tumors, i.e. UL. UL not only are known to show rarely if ever mutational inactivation of p53 (Baek et al., Am. J. Obstet. Gynecol. 188 (2003), 634-639; Martel et al., Semin Reprod. Med. 22 (2004), 91-103) but apparently show a growth behaviour that depends on the p19Arf-MDM2-p53 autoregulation. As to the effects induced by nutlin-3 it seems particular noteworthy that the cells are not resistant to p53-induced apoptosis as are fibroblasts of both human and murine origin (Efeyan et al., 2007). The BAX-p19Arf correlation as demonstrated in samples from native fibroids in the examples of the present invention shows that in vivo leiomyoma cells are not resistant to p53-dependent apoptosis as well. Of note, Bax protein has been shown to be detectable in leiomyoma samples but not in myometrial samples (Wu et al., J. Steroid Biochem. Mol. Biol. 80 (2002), 77-83). In summary, the mechanisms limiting the in vitro proliferation of UL cells at least in some aspects seem to reflect an endogenous growth control of UL in vivo. Self renewal induced by HMGA2 seems to be a positive regulator of UL growth while the elements of an intact p19Arf-driven network assure the high genomic stability of UL as well as their growth control. In vitro antagonizing MDM2 induces cellular senescence as apoptosis and offers promising new approaches towards a therapy of UL.

### Example 4

### Fibroid Explants Reveal a Higher Sensitivity Against MDM2 Inhibitor Nutlin-3 than Matching Myometrium

In the following, the question was addressed whether fibroids display a higher sensitivity than matching myometrium as can be suggested from their higher expression of p14^{Arf} as shown herein (cf. [7,9]). Therefore, tissue explants were used taken from leiomyomas and matching myometrium to analyze the effects of an MDM2 antagonist and possible different sensitivities of the fibroids and their matching tissue of origin.

Nutlin-3 Induces Senescence as Well as Apoptosis in Tissue Explants from Leiomyomas

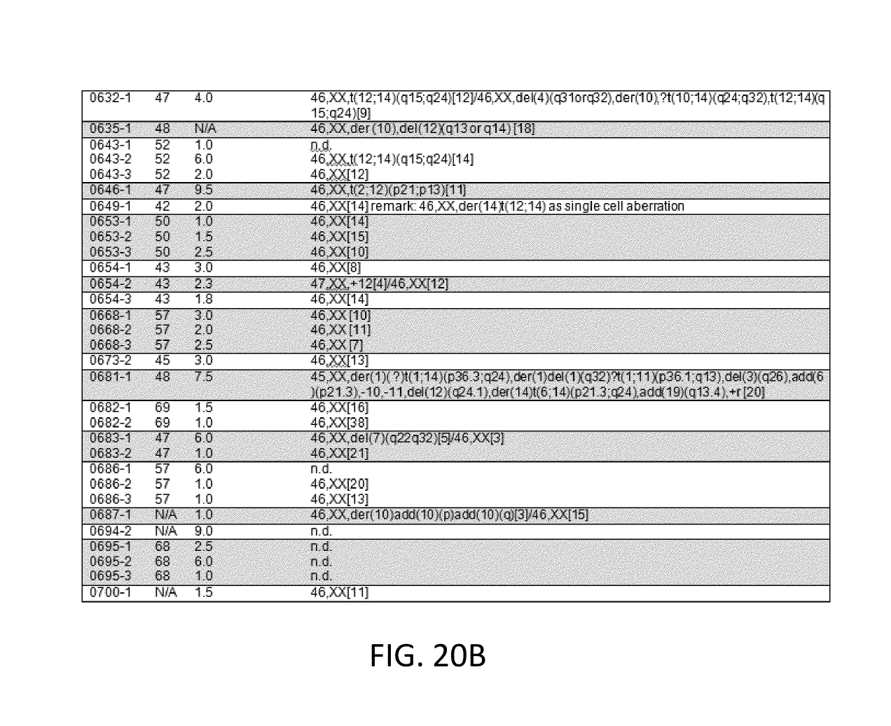

To investigate whether nutlin-3 can induce similar effects on the gene expression of p21 and BAX as observed above, explants from a total of four leiomyomas (Table 1 in FIGS. 20A and 20B) were incubated with nutlin-3 (3 μM and 10 μM each) for 72 h and then checked for the expression of these genes. In addition, the expression of the proliferation marker Ki-67 was analyzed. Compared to the controls the results show concentration-dependent highly significant (p<0.01 or p<0.001, respectively) increases of the expression of p21 and BAX in all explants analyzed (FIG. 10 A, B) with both concentrations used. Accordingly, the expression of Ki-67 strongly decreased except for one case where with a concentration of 3 μM an increase of Ki-67 expression was noted (FIG. 10 C).

### Nutlin-3 Increases the Amount of p53 in a Concentration-Dependent Manner

Nutlin-3 acts as an inhibitor of MDM2 which in turn destabilizes p53. To see whether the increased expression of the senescence and apoptotic markers noted after treatment of the explants with nutlin-3 was indeed due to an elevated level of p53, explants of case 0700-1 treated with 30 μM and 50 μM nutlin-3, respectively, for 72 h were used for immunoblot analyses. The results clearly show a concentration-dependent increase of p53 (FIG. 11A, B).

Fibroids Express Significantly Higher Levels of p14^{Arf} than Matching Myometrium

Expression analysis of a series of UL and eight myometrial samples revealed a significantly higher expression of p14^{Arf} in the UL compared to their tissue of origin, as shown in Examples 1 to 3 and in [7]. This smaller series has herein been extended by a second set of experiments to a total of 52 fibroids and 31 matching myometrial tissues. On average, the myometrial samples expressed 10-fold lower levels of p14^{Arf} than the fibroids (1.14 vs. 11.5) (p<0.001). Moreover, in all but two fibroids the expression of p14^{Arf} in the fibroid taken from a patient exceeded that of the corresponding myometrium (FIG. 12). A marked heterogeneity of the expression between the UL of individual patients was noted as well.

As a Rule Leiomyomas Show a Higher Sensitivity Against Nutlin-3 Treatment than Matching Myometrium

As disclosed herein and in [7], leiomyomas show elevated levels of p14^{Arf} mRNA compared to myometrium resulting from a negative feedback-loop between p53 and MDM2 which in turn results in an activation of MDM2. Furthermore, without intending to be bound by theory, it was hypothesized herein and in [11] that within the p14^{Arf}-driven pathway a delicate balance between p53 and MDM2 assures the proliferative activity of leiomyomas as well as their genomic integrity. Accordingly, it can be speculated that leiomyomas may be more sensitive against MDM2 inhibition than matching myometrium. Therefore, samples of myometrial tissue from the patients investigated before and from one additional case (one leiomyoma and its matching myometrium; cf. Table 1 in FIGS. 20A and 20B) have been treated with 3 μM and 10 μM nutlin-3 for 72 h in a way to analyze and compare the expression of p21, and BAX with that revealed by the matching leiomyomas.

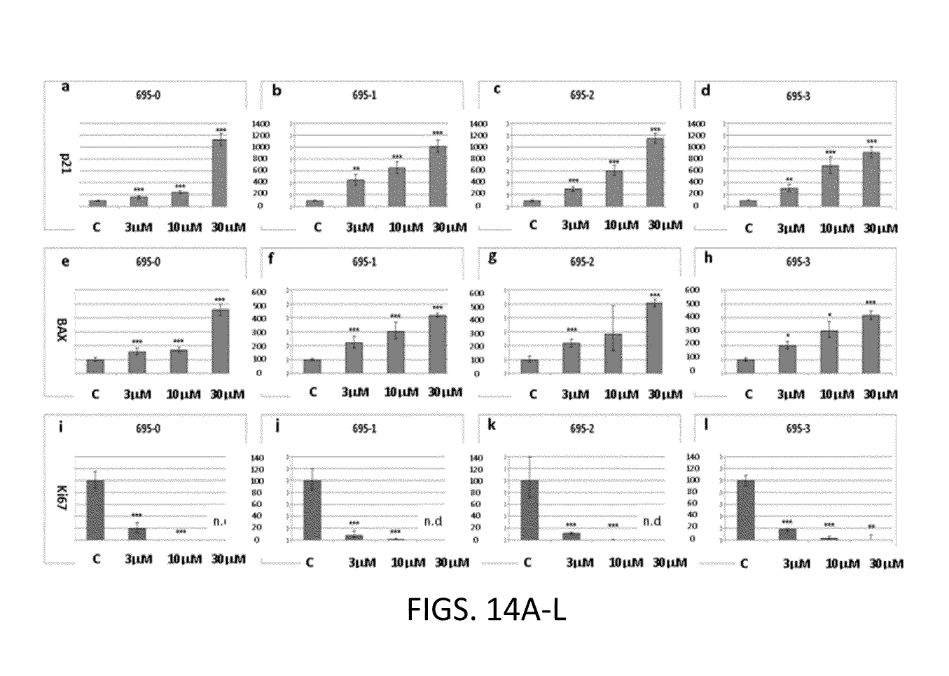

As to the expression of p21, in all tissue explants treated the p21 expression in the fibroids exceeded that seen in the matching myometrial tissue indicating a higher sensitivity (FIG. 13 A). Similarly, in case of BAX a generally higher sensitivity of the leiomyoma explants was noted except for two cases (683 and 687) where due to the addition of 10 μM nutlin-3 the expression in the myometrium exceeded that of the leiomyoma (FIG. 13 B).

### Nutlin-3 Treatment Induces an Increasing Amount of p53

Because MDM2 inhibition can be expected to raise the level of p53 and it was shown that nutlin-3 treatment increases the amount of p53 expressed by the explants (c.f. FIGS. 11A and 11B) we were interested to see if an IHC-scoring system (scoring system based on the staining intensity of immunohistochemically stained tissues) may also detect the comparable differences between UL and matching myometrium as those shown by qRT-PCR for the expression p21 and BAX. Treatment of the explants by nutlin-3 caused a clearly increased intensity of p53 staining which was concentration-dependent (Table 2). Further analyses showed an increased number of p53-positive cells as well when comparing UL with matching myometrium.

TABLE 2
Treatment by nutlin-3 results in a concentration dependent increase of the
number of p53 positive cells as well as of the staining intensity as revealed
by immunohistochemistry. For further information of the tumors studied
(karyotype, age, tumor size) see Table 1 in FIGS. 20A and 20B.

| | | duration of | number of p53- | |
|---|---|---|---|---|
| #case | treatment | treatment | positive cells | intensity |
| 0694-2 | control | 72 h | 11 | 0-1 |
| | 10 μM nutlin-3 | | 393 | 2 |
| | 30 μM nutlin-3 | | 1,277 | 3 |
| 0695-0 | control | 6 days | 0 | 0 |
| | 10 μM nutlin-3 | | 11 | 1 |
| | 30 μM nutlin-3 | | 188 | 1 |
| 0695-1 | control | | 0 | 0 |
| | 10 μM nutlin-3 | | 42 | 0-1 |
| | 30 μM nutlin-3 | | 89 | 1 |
| 0695-2 | control | | 0 | 0 |
| | 10 μM nutlin-3 | | 194 | 1-2 |
| | 30 μM nutlin-3 | | 799 | 3 |
| 0695-3 | control | | 0 | 0 |
| | 10 μM nutlin-3 | | 770 | 3 |
| | 30 μM nutlin-3 | | 1,493 | 3 |
| 0687-0 | control | 72 h | 0 | 0 |
| | 10 μM nutlin-3 | | 2 | 1 |
| | 30 μM nutlin-3 | | 257 | 2 |
| 0687-1 | control | | 0 | 0 |
| | 10 μM nutlin-3 | | 398 | 2 |
| | 30 μM nutlin-3 | | n.d. | n.d. |

### Uterine Leiomyoma Cells do not Recover During Long-Term Inhibition of MDM2

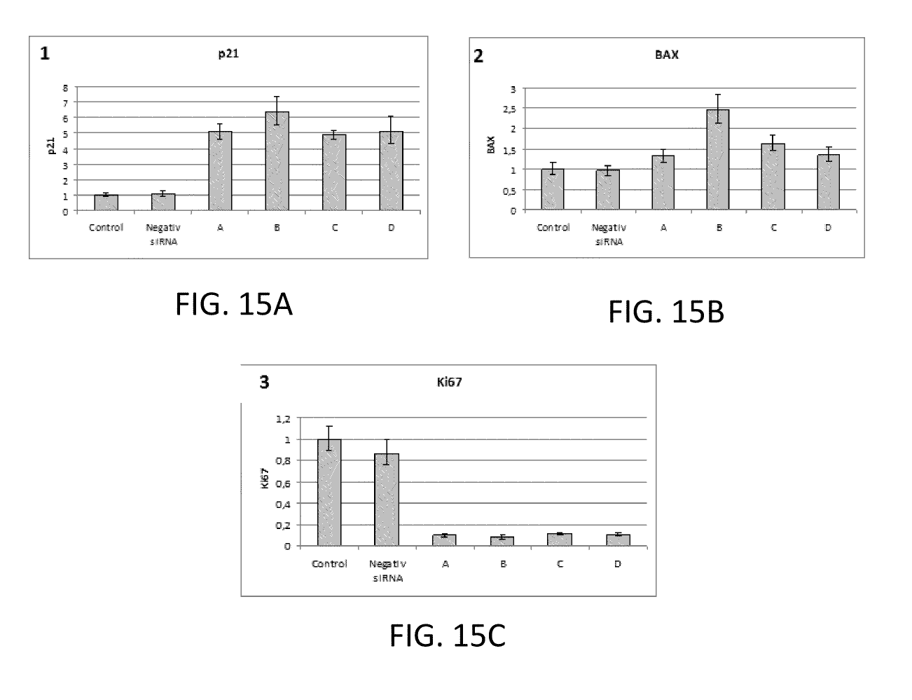

To see how the gene expression patterns for p21, BAX, and Ki-67 change after six days of MDM2 inhibition and whether the cells become resistant, additional gene expression analyses for these genes on a series of three UL along with matching myometrium were performed (FIGS. 14A-L). Akin to a shorter exposure to nutlin-3, the long term experiment led to a significant increase in the expression of p21 (FIGS. 14A-D) and BAX (FIGS. 14E-H), and to a decreased expression of Ki-67 (FIGS. 14I-L) in a dose-dependent manner. Furthermore, clear differences between the myometrium and any of the matching fibroids became apparent pointing again at a reduced sensitivity of the myometrium to the MDM2 inhibition.

### Discussion

As shown above in Example 1 and in [7], leiomyomas express higher levels of p14^{Arf}, an important upstream regulator of p53 compared to myometrium. Herein, this observation has been confirmed on a much larger series of cases. In addition, a marked heterogeneity of the p14^{Arf} expression was noted when comparing UL from one patient. Most likely due to the well-documented positive feedback-loop that exists between p53 and MDM2 [12, 13], the fibroids do also express higher levels of MDM2 leading to a positive correlation between p14^{Arf} and MDM2 expression [7] that may keep fibroids in a delicate balance between growth and senescence. This makes MDM2 an interesting target molecule for the growth control of leiomyomas. Accordingly, as demonstrated herein by the inventors, in vitro treatment of leiomyoma cells with nutlin-3, a small-molecule inhibitor of MDM2, activates the expression of canonical groups of genes associated with senescence and apoptosis downstream of p53 (Markowski et al., submitted for publication). Herein, it is shown that as a rule tissue explants taken from leiomyomas display a higher sensitivity after treatment with nutlin-3 than do those from matching myometrium. Interestingly, nutlin-3 increased the expression of p53-dependent marker genes associated with senescence as well as with apoptosis. As a rule for both genes, i.e. p21 and BAX, leiomyoma tissue turned out to be more sensitive than myometrial tissue. It is assumed that the higher expression of p14^{Arf} in the fibroids, as shown herein and in [7], is the most likely explanation for this different sensitivity. When interpreting leiomyomas as the result of proliferation of a stem-cell like population it seems reasonable to assume that this proliferation is accompanied by activation of the p53-pathway via p14^{Arf} to protect the cellular genome [11]. Vice versa, functional p53 activates the positive feedback-loop with MDM2 that may be essential for the development of fibroids. Likewise, disturbing this balance by MDM2 inhibitors can be assumed to cause senescence as well as apoptosis of the leiomyoma cell population because p53 still remains intact with a strongly decreased MDM2 activity.

Of course, the use of an in vitro model has some limitations. Cell cultures from fibroids can easily be set up without major problems of overgrowing of normal cells but the cells have a limited growth potential only and, as an even more serious reason for concern, rapidly lose characteristic features of leiomyomas in vivo. For example, the estrogen receptor level of fibroid cells rapidly declines in vitro [14]. Immortalization of the cells is possible, e.g., by using SV40-large T-antigen or the SV40 early region, but experiments with these cells are still facing the problem of drastic changes compared to the normal cells [15]. Though the explant cultures as used herein may reflect the in vivo situation better than isolated cells, it is well known that explant cultures lose characteristics of the tissue in the body's natural environment as well, e.g., a decrease in the expression of estrogen receptors after a short time in vitro has been noted [16] that was within the time-range used here for the nutlin-3 experiments. Nevertheless, the higher sensitivity of the leiomyoma tissue against the inhibition of MDM2 compared to surrounding myometrium corresponds to a higher in vivo expression of p14^{Arf} and thus likely exists in vivo as well. Therefore, it has considerable therapeutical implications as well. In UL antagonizing MDM2 seems to be a way to induce growth arrest as well as apoptosis. Both can be expected to irreversibly impair tumor growth and to decrease the tumor size, respectively. Interestingly, estrogens are known as negative regulators of p53 [17]. Thus, it seems reasonable to speculate that changes of the behaviour of fibroids following changes of the hormonal milieu as in particular their shrinkage are at least in part also due to skewing the balance towards p53. Accordingly, a combination of, e.g., a GnRH antagonist and an MDM2 antagonist is a favourable approach for the treatment of fibroids. In summary, the results provided by the present invention validate the idea that senescence and apoptosis play an important role in the growth control of fibroids and that their induction offers interesting approaches for the therapy of these frequent tumors.

### Additional Materials and Methods

### Tissue Samples

Altogether, tissue samples of 36 patients have been investigated herein; see Table 1 in FIGS. 20A and 20B. The study was approved by the local ethics committee and prior to surgery; informed written consent was obtained from all patients. For gene expression studies samples of 52 UL from 31 patients along with matching myometrium were taken during surgery, immediately frozen in liquid nitrogen, and stored at −80° C. for RNA isolation and qRT-PCR analyses of p14^{Arf}. For MDM2 inhibition six tissue samples of UL from four patients as well as matching myometrium were taken during surgery and immediately transferred into sterile Hank's solution.

Treatment with Nutlin-3

For treatment with nutlin-3 (Biomol, Hamburg, Germany) tissue samples were minced into small pieces of approximately 0.5 cm diameter and incubated in medium 199 supplemented with 20% FCS and nutlin-3 (3, 10, or 50 μM) for 72 h. As controls tissue explants were incubated in medium 199 supplemented with 20% FCS without nutlin-3 for 72 h.

cDNA-Synthesis

About 250 ng of total RNA were reverse transcribed with 200 U/μl of M-MLV reverse transcriptase (Invitrogen, Karlsruhe, Germany), RNase Out, 150 ng random hexamers and 10 mM dNTPs according to the manufacturer's instructions. RNA was denatured at 65° C. for 5 min and subsequently kept on ice for 1 min. After adding the enzyme to the RNA primer mixes, samples were incubated for 10 min at 25° C. to allow annealing of the random hexamers. Reverse transcription was performed at 37° C. for 50 mM followed by inactivation of the reverse transcriptase at 70° C. for 15 min.

Quantitative Real-Time PCR (qRT-PCR)

Relative quantification of transcription levels was carried out by real-time PCR analyses using the Applied Biosystems 7300 real-time PCR system (Applied Biosystems, Darmstadt, Germany). Commercially available gene expression assays (Applied Biosystems, Darmstadt, Germany) were used for quantification of mRNA of human p14^{Arf} (Hs00924091), BAX (Hs00180269_m1), CDKN1A (Hs 99999142), and MKI67 (Hs00606991_m1). HPRT served as endogenous control [7]. All qRT-PCR experiments were done in triplicate.

### Immunohistochemical Studies of Tissue Explants

The immunohistochemical staining for the p53 protein (clone DO-7, DAKO, Glostrup, Denmark) was performed using a detection kit (DAKO ChemMate, DAKO, Glostrup, Denmark) and a semi-automated stainer (DAKO TechMate, DAKO, Glostrup, Denmark) according to the specifications of the manufacturer. For antigen retrieval the slides were treated in a PT Link module (DAKO, Glostrup, Denmark) using the EnVision™ FLEX Target Retrieval Solution, Low pH (DAKO, Glostrup, Denmark). The antibody dilution was 1:100.

### Western Blot Analysis

Protein extracts were obtained using RIPA buffer and concentrations were determined using a BCA protein assay (Thermo Scientific, Rockford, USA) according to the manufacturer's instructions. Total protein (16 μg per lane) was separated using a SDS-polyacrylamide gel and transferred onto a nitrocellulose membrane. SeeBlue Plus2 Pre-Stained Standard (Invitrogen, Karlsruhe, Germany) was used as marker. The membranes were incubated with primary anti-p53 (mouse, monoclonal, 1:200; DAKO, Hamburg, Germany) and anti-beta-actin (rabbit, polyclonal, 1:7,500; Santa Cruz Biotechnology, California, USA) followed by incubation with the corresponding secondary antibodies (alkaline phosphatase-conjugated goat anti-mouse IgG (1:5,000) (Invitrogen, Karlsruhe, Germany) and alkaline phosphatase-conjugated bovine anti-rabbit IgG (1:3,750) (Santa Cruz Biotechnology, California, USA)). The bands were visualized by incubating the membrane with NBT/BCIP (Nitro blue tetrazolium chloride/5-Bromo-4-chloro-3-indolyl phosphate) (Roche Applied Science, Mannheim, Germany) according to the manufacturer's instructions Immunoblots were scanned using a flatbed scanner and image analysis was performed with the ImageJ gel analysis algorithm (Rasband, W. S., ImageJ, U.S. National Institutes of Health, Bethesda, Md., USA, http://rsb.info nih gov/ij/, 1997-2009; Abramoff et al., Biophotonics Int. 11 (2004), 36-42).

### Example 5

### Treatment of Cells from Fibroids In Vitro by siRNA Antagonizing MDM2

The question was addressed whether a specific downregulation of MDM2 by appropriate siRNAs can reduce the growth potential of cells from uterine leiomyomas and at the same time increase the expression of two genes linked to senescence and apoptosis, respectively.

Treatment of the cells by siRNAs specifically designed against MDM2 leads to an increased expression of p21 (FIG. 15A) and BAX (FIG. 15B) and to a decreased expression of Ki-67 (FIG. 15C) validating this approach as a mean to reduce the growth potential of cells from uterine leiomyomas by increasing the expression of genes linked to senescence and apoptosis, i.e. p21 and BAX.

### Additional Materials and Methods

### Cell Culture

Tissue samples of fibroids stored in sterile Hank's solution were minced into small pieces followed by treatment with 0.26% (200 U/ml) collagenase (Serva, Heidelberg, Germany). After 1-2 h, the dissociated cells were transferred into sterile 25 cm² cell culture flasks containing 5 ml medium 199, supplemented with 20% fetal bovine serum (FCS, Invitrogen, Karlsruhe, Germany) and 2% penicillin-streptomycin (Biochrom, Berlin, Germany). The cultures were incubated in 5% CO₂ air at 37° C. and medium was changed every 2-3 days. Cultures were passaged when reaching 80% confluence using 1×TrypLE Express in a PBS-EDTA buffer.

### RNA Interference

Cells were transfected with four MDM2-specific siRNAs (SIH900207ABCD, SABiosciences, Frederick, USA) by SureFECT transfection reagent (SABiosciences, Frederick, USA) by reverse transfection following the manufacturer's instruction. Cells were harvested, suspended in medium 199 supplemented with 20% FCS and divided into six groups which were treated with the four different MDM2-siRNAs, a nonspecific siRNA (SABiosciences, Frederick, USA) as negative control, and SureFECT vehicle only. For each transfection, a 2,200 μL cell suspension containing 200,000 cells was transfected with 2 μM siRNA using 6 μL transfection reagent in 6-well plates. Cells were kept under normal culture conditions and were harvested 48 h after transfection for qRT-PCR analysis.

Quantitative Real-Time PCR (qRT-PCR)

Relative quantification of transcription levels was carried out by real-time PCR analyses using the Applied Biosystems 7300 real-time PCR system (Applied Biosystems, Darmstadt, Germany). Commercially available gene expression assays (Applied Biosystems, Darmstadt, Germany) were used for quantification of mRNA of human BAX (Hs00180269_m1), p21 (Hs99999142), and Ki-67 (Hs00606991_m1). HPRT served as endogenous control [7]. All qRT-PCR experiments were done in triplicate.

### Example 6

### Treatment of Cells and Explants from Fibroids In Vitro by the MDM2 Antagonist RITA (Reactivation of p53 and Induction of Tumor Cell Apoptosis)

In the following, the question is addressed whether akin to the treatment with nutlin-3 the small-molecule MDM2 inhibitor RITA (Reactivation of p53 and Induction of Tumor cell Apoptosis/2,5-bis-(5-Hydroxymethyl-2-thienyl)-furan (NSC 652287)) can reduce the growth potential of cells and explants from uterine leiomyomas and at the same time increase within said cells the expression of two genes linked to senescence and apoptosis, respectively.

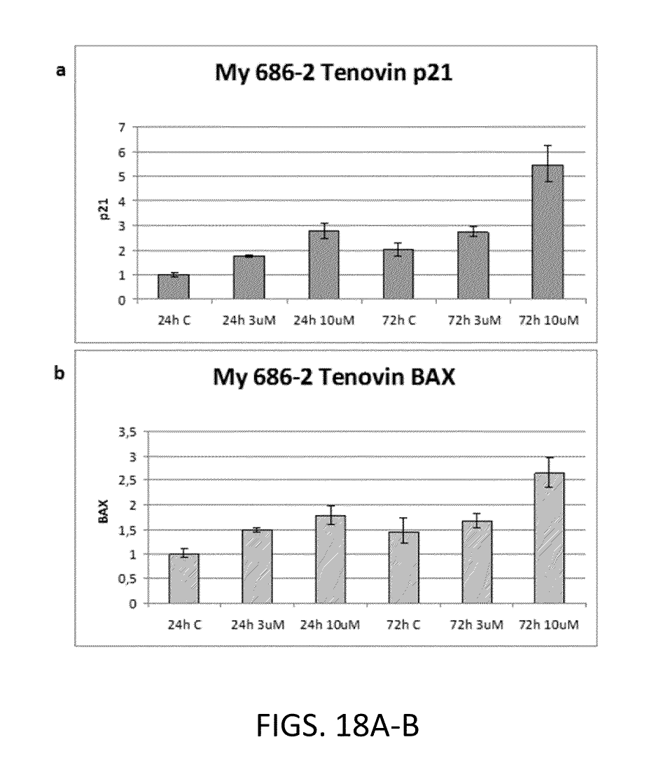

In cells of fibroids in vitro treatment by 10 μM RITA caused a significant increase of the expression of p21 (FIG. 16A), β-galactosidase (FIG. 16B), and BAX (FIG. 16C) as well as a decrease of the expression of Ki-67 (FIG. 16D). As a next step explants of fibroids and matching myometrium in short term culture were treated with RITA and a significant increase of the expression of p21 (FIG. 17A) and of BAX (FIG. 17B) was noted attesting a similar potential to irreversibly impair tumor growth and decrease the tumor size of RITA as shown above for nutlin-3.

### Additional Materials and Methods

### Tissue Samples

For the experiments explants from a leiomyoma with a clonal complex chromosomal rearrangement involving chromosome 10 along with matching myometrium were taken during surgery and immediately transferred into sterile Hank's solution.

Treatment of Explants with RITA

For treatment with RITA (Biomol, Hamburg, Germany) tissue samples were minced into small pieces of approximately 0.5 cm diameter and incubated in medium 199 supplemented with 20% FCS and RITA (10 μM) for 72 h. As controls tissue explants were incubated in medium 199 supplemented with 20% FCS without RITA for 72 h.

Treatment of Leiomyoma Cells with RITA

For treatment with RITA (Biomol, Hamburg, Germany) cells were plated in Leighton-tubes at a density of 200,000 on 10 mm×50 mm cover slips 24 h before incubation with RITA. Cells were treated with 1 μM and 10 μM RITA for 72 h. As controls cells were cultured in medium 199 supplemented with 20% FCS without RITA for 72 h.

Quantitative Real-Time PCR (qRT-PCR)

Relative quantification of transcription levels was carried out by real-time PCR analyses using the Applied Biosystems 7300 real-time PCR system (Applied Biosystems, Darmstadt, Germany). Commercially available gene expression assays (Applied Biosystems, Darmstadt, Germany) were used for quantification of mRNA of human BAX (Hs00180269_m1), p21 (Hs99999142), β-Gal (Hs01035162) and Ki67 (Hs00606991_m1). HPRT served as endogenous control [7]. All qRT-PCR experiments were done in triplicate.

### Example 7

### Treatment of Cells from Fibroids In Vitro by the MDM2 Antagonist Tenovin-1

In the following, the question is addressed whether akin to the treatment with nutlin-3 the small-molecule MDM2 inhibitor Tenovin-1 can reduce the growth potential of cells from uterine leiomyomas and at the same time increase the expression of two genes linked to senescence and apoptosis, respectively.

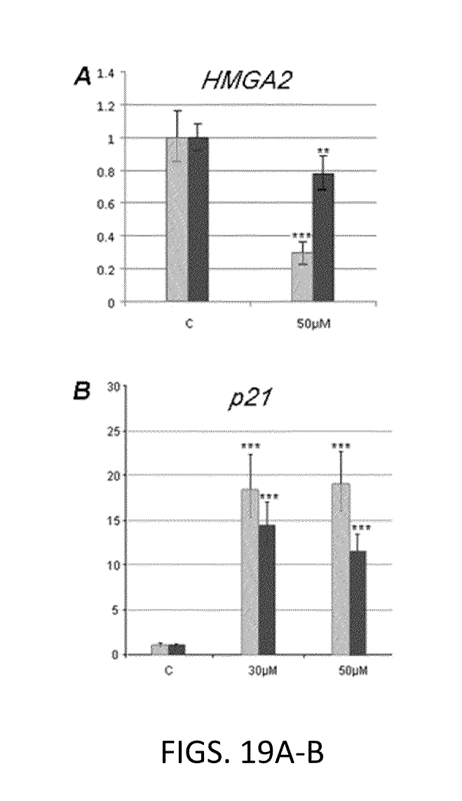

In vitro treatment with Tenovin-1 caused a significant increase of the expression of p21 (FIG. 18A) and BAX (FIG. 18B) in cells of fibroids, showing thus, in combination with the above described experiments testing different MDM2 antagonist the general potential of compounds with MDM2 antagonistic properties for use in the treatment of diseases as defined hereinabove.

### Additional Materials and Methods

Treatment of Leiomyoma Cells with Tenovin-1

For treatment with Tenovin-1 (Biomol, Hamburg, Germany) cells were plated in Leighton-tubes at a density of 200,000 on 10 mm×50 mm cover slips 24 h before incubation with Tenovin-1. Cells were treated with 3 μM and 10 μM Tenovin-1 for 24 and for 72 h, respectively. As controls cells were cultured in medium 199 supplemented with 20% FCS without Tenovin-1 for 24 and for 72 h, respectively.

### Example 8

### Effects of Treatment of Adipose-Tissue Derived Stem/Progenitor Cells (ADSC) by Nutlin-3 on the Expression of High Mobility AT-Hook 2 (HMGA2) and on the Expression of p21

In the following, a possible downregulation of the HMGA2 gene by treatment of ADSCs with the small-molecule MDM2 inhibitor nutlin-3 was investigated. HMGA2 is associated with the self-renewal capacity of somatic stem cells. Furthermore, it was tested whether this treatment caused an increased expression of p21 associated with cellular senescence.

Treatment of ADSCs by nutlin-3 caused a significant decrease of the expression of HMGA2 (FIG. 19A) and an increase of the expression of p21 (FIG. 19B). As mentioned above, it is not known, whether the cells of UL origin from stem cell like cells of the myometrium cell or from more differentiated cells. Both however, ADSCs and cells from uterine leiomyomas are of mesenchymal origin. Comparable to the effect on UL cells, treatment with MDM2 antagonists led to an decreased self-renewal capacity of the ADS cells as well, pointing at a general usefulness of the methods and compounds of the present invention in the treatment of diseases characterized by an aberrant growth of mesenchymal stem cells and their descendants. Thus, diseases such as endometriosis, adenomyosis, endometrial hyperplasia, leiomyoma, lipoma, hamartoma of the lung, fibroadenoma of the breast, adenoma of the salivary gland, and aggressive angiomyxomas may also be treated by the methods of the present invention. Accordingly, obesity, as a disorder characterized by an overgrowth of the adipose tissue may be treated according to the methods of the present invention as well.

Therefore, nutlin-3 and other MDM2 antagonists such as antibodies, siRNA, microRNA, aptamers, and spiegelmers or small molecules such as nutlins, tenovins, tenovin-1, tenovin-6, or RITA may be used to decrease the self-renewal capacity of somatic stem cells, in particular of adipose-tissue, for use in the treatment of disorders associated with an overgrowth of adipose-tissue, i.e. obesity.

### Additional Materials and Methods

### Isolation and Culture of Canine Adipose Tissue Derived Stem Cells (ADSCs)

Canine subcutaneous adipose tissue was taken during surgery, transferred into sterile Hank's solution and minced for cell culture into small pieces followed by treatment with 0.26% (200 U/ml) collagenase (Serva, Heidelberg, Germany). From these samples ADSCs were isolated. Briefly, after 1-2 hours, the dissociated cells were transferred into sterile 25 cm² cell culture flasks containing 5 ml medium 199 (Gibco, Karlsruhe, Germany), supplemented with 20% fetal bovine serum (FCS, Invitrogen, Karlsruhe, Germany) and 2% penicillin-streptomycin (Biochrom, Berlin, Germany). The cultures were incubated in 5% CO₂ air at 37° C. and medium was changed every two to three days. Cultures were passaged when reaching 80% confluence using 1×TrypLE Express (Gibco, Karlsruhe, Germany) in a PBS-EDTA buffer. Cells were subcultured in medium 199 with 10% FCS and antibiotics.

Treatment with Nutlin-3

For treatment with nutlin-3 canine ADSCs were plated in Leighton-tubes at a density of 200,000 on 10 mm×50 mm cover slips 24 h before incubation with nutlin-3 (Biomol, Hamburg, Germany). Cells were treated with 30 μM and 50 μM nutlin-3 for 72 h. As controls cells were cultured in medium 199 supplemented with 20% FCS without nutlin-3 for 72 h.

### Quantitative Real-Time PCR

Relative quantification of transcription levels was carried out by real-time PCR analyses using the Applied Biosystems 7300 Real-Time PCR system (Applied Biosystems, Darmstadt, Germany). For quantification of mRNA levels of canine p21a commercially available gene expression assay was used (Hs99999142) (Applied Biosystems, Darmstadt, Germany). Primers and probe used to amplify canine HMGA2 were 5′-AGT CCC TCC AAA GCA GCT CAA AAG-3′ (forward) (SEQ ID NO.:4), 5′-GCC ATT TCC TAG GTC TGC CTC-3′ (reverse) (SEQ ID NO.:5), and 5′-6-FAM-GAA GCC ACT GGA GAA AAA CGG CCA-TAMRA-3′ (SEQ ID NO.:6) (probe). For canine cells the housekeeping gene beta-glucuronidase (GUSB) served as the reference for relative quantification because in contrast to some other housekeeping genes its expression turned out not to increase in serum starved ADSCs when induced by fetal serum in our own experiments. Moreover, in a paper by Gorzelniak et al. (2001) it was shown that in preadipocytes GUSB mRNA remained relatively constant when they were not subjected to differentiation towards mature adipocytes. To amplify the GUSB mRNA the primers 5′-TGG TGC TGA GGA TTG GCA-3′ (forward) (SEQ ID NO.:7), 5′-CTG CCA CAT GGA CCC CAT TC-3′ (reverse) (SEQ ID NO.:8) and probe 5′-6-FAM-CGC CCA CTA CTA TGC CAT CGT GTG T-TAMRA-3′ (SEQ ID NO.:9) were used. All qRT-PCR experiments were done in triplicate.

### REFERENCES

1. Cramer S F, Patel A: The frequency of uterine leiomyomas. Am J Clin Pathol 1990, 94:435-438.
2. Baird D D, Dunson D B, Hill M C, Cousins D, Schectman J M: High cumulative incidence of uterine leiomyoma in black and white women: ultrasound evidence. Am J Obstet Gynecol 2003, 188:100-107.
3. Chia C C, Huang S C, Chen S S, Kang J Y, Lin J C, Lin Y S, Huang K F, Lee H J, Zheng C C: Ultrasonographic evaluation of the change in uterine fibroids induced by treatment with a GnRH analog. Taiwan J Obstet Gynecol 2006, 45:124-128.
4. Matta W H, Shaw R W, Nye M: Long-term follow-up of patients with uterine fibroids after treatment with the LHRH agonist buserelin. Br J Obstet Gynaecol 1989, 96:200-206.
5. Lethaby A, Vollenhoven B, Sowter M: Pre-operative GnRH analogue therapy before hysterectomy or myomectomy for uterine fibroids. Cochrane Database Syst Rev 2001, CD000547.
6. Miller C E: Unmet therapeutic needs for uterine myomas. J Minim Invasive Gynecol 2009, 16:11-21.
7. Markowski D N, von Ahsen I, Nezhad M H, Wosniok W, Helmke B M, Bullerdiek J: HMGA2 and the p14Arf-TP53-CDKNIA Axis: A Delicate Balance in the Growth of Uterine Leiomyomas. Genes Chromosomes Cancer 2010a, 49:661-668.
8. Lev Bar-Or R, Maya R, Segel L A, Alon U, Levine A J, Oren M: Generation of oscillations by the p53-Mdm2 feedback loop: a theoretical and experimental study. Proc Natl Acad Sci USA 2000, 97:11250-11255.
9. Laser J, Lee P, Wei J J: Cellular senescence in usual type uterine leiomyoma. Fertil Steril 2010, 93:2020-2026.
10. Klemke M, Meyer A, Nezhad M H, Bartnitzke S, Drieschner N, Frantzen C, Schmidt E H, Beige G, Bullerdiek J: Overexpression of HMGA2 in uterine leiomyomas points to its general role for the pathogenesis of the disease. Genes Chromosomes Cancer 2009, 48:171-178.
11. Markowski D N, Helmke B M, Bullerdiek J: Cellular senescence in usual type uterine leiomyoma. Fertil Steril 2010b, 94:e79; author reply e80.
12. Jolma I W, Ni X Y, Rensing L, Ruoff P: Harmonic oscillations in homeostatic controllers: Dynamics of the p53 regulatory system. Biophys J 2010, 98:743-752.
13. Proctor C J, Gray D A: Explaining oscillations and variability in the p53-Mdm2 system. BMC Syst Biol 2008, 2:75.
14. Zaitseva M, Vollenhoven B J, Rogers P A: In vitro culture significantly alters gene expression profiles and reduces differences between myometrial and fibroid smooth muscle cells. Mol Hum Reprod 2006, 12:187-207.
15. Stern C, Kazmierczak B, Thode B, Rommel B, Bartnitzke S, Dal C M P, van de Ven W, Van den Berghe H, Bullerdiek J: Leiomyoma cells with 12q15 aberrations can be transformed in vitro and show a relatively stable karyotype during precrisis period. Cancer Genet Cytogenet 1991, 54:223-228.
16. Severino M F, Murray M J, Brandon D D, Clinton G M, Burry K A, Novy M J: Rapid loss of oestrogen and progesterone receptors in human leiomyoma and myometrial explant cultures. Mol Hum Reprod 1996, 2:823-828.
17. Gao Z, Matsuo H, Nakago S, Kurachi 0, Maruo T: p53 Tumor suppressor protein content in human uterine leiomyomas and its down-regulation by 17 beta-estradiol. J Clin Endocrinol Metab 2002, 87:3915-3920.
18. Shaffer L G, Slovak M L, Campbell L J, editors ISCN: In: An International System for Human Cytogenetic Nomenclature 2009, Basel, Switzerland: S. Karger.
19. Asada H, Yamagata Y, Taketani T, Matsuoka A, Tamura H, Hattori N, Ohgane J, Hattori N, Shiota K, Sugino N. 2008. Potential link between estrogen receptor-alpha gene hypomethylation and uterine fibroid formation. Mol Hum Reprod. 14:539-45.
20. Atkins K A, Arronte N, Dams C J, Rice L W. 2008. The use of p16 in enhancing the histologic classification of uterine smooth muscle tumors. Am J Surg Pathol. 32:98-102.
21. Bullerdiek J. 1999. Leiomyoma—do viruses play the main role? Genes Chromosomes Cancer. 26:181.
22. Carney S A, Tahara H, Swartz C D, Risinger J I, He H, Moore A B, Haseman J K, Barrett J C, Dixon D. 2002 Immortalization of human uterine leiomyoma and myometrial cell lines after induction of telomerase activity: molecular and phenotypic characteristics. Lab Invest. 82:719-28.
23. Chang H L, Senaratne T N, Zhang L, Szotek P P, Stewart E, Dombkowski D, Preffer F, Donahoe P K, Teixeira J. 2009. uterine leiomyomas exhibit fewer stem/progenitor cell characteristics when compared with corresponding normal myometrium. Reprod Sci. 2009 Oct. 5. [Epub ahead of print]
24. Gross K L, Neskey D M, Manchanda N, Weremowicz S, Kleinman M S, Nowak R A, Ligon A H, Rogalla P, Drechsler K, Bullerdiek J, Morton C C. 2003. HMGA2 expression in uterine leiomyomata and myometrium: quantitative analysis and tissue culture studies. Genes Chromosomes Cancer. 38:68-79.
25. Hennig Y, Deichert U, Bonk U, Thode B, Bartnitzke S, Bullerdiek J. 1999. Chromosomal translocations affecting 12q14˜15 but not deletions of the long arm of chromosome 7 associated with a growth advantage of uterine smooth muscle cells. Mol Hum Reprod. 5:1150-4
26. Lee C H, Turbin D A, Sung Y C, Espinosa I, Montgomery K, van de Rijn M, Gilks C B. 2009. A panel of antibodies to determine site of origin and malignancy in smooth muscle tumors. Mod Pathol. 22:1519-31.
27. Li O, Li J, Dröge P. 2007. DNA architectural factor and proto-oncogene HMGA2 regulates key developmental genes in pluripotent human embryonic stem cells. FEBS Lett. 581:3533-7.
28. Li O, Vasudevan D, Davey C A, Dröge P. 2006. High-level expression of DNA architectural factor HMGA2 and its association with nucleosomes in human embryonic stem cells. Genesis. 44:523-9.
29. Ligon A H, Morton C C. 2000. Genetics of uterine leiomyomata. Genes Chromosomes Cancer. 28:235-45.
30. Lowe S W, Sherr C J. 2003. Tumor suppression by Ink4a-Arf: progress and puzzles. Curr Opin Genet Dev. 13:77-83.
31. Loy C J, Evelyn S, Lim F K, Liu M H, Yong E L. 2005. Growth dynamics of human leiomyoma cells and inhibitory effects of the peroxisome proliferator-activated receptor-gamma ligand, pioglitazone. Mol Hum Reprod. 11:561-6.
32. Luo X, Chegini N. 2008. The expression and potential regulatory function of microRNAs in the pathogenesis of leiomyoma. Semin Reprod Med. 26:500-14
33. Marsh E E, Lin Z, Yin P, Milad M, Chakravarti D, Bulun S E. 2008. Differential expression of microRNA species in human uterine leiomyoma versus normal myometrium. Feral Steril. 89:1771-6.
34. McKeller R N, Fowler J L, Cunningham J J, Warner N, Smeyne R J, Zindy F, Skapek S X. 2002. The Arf tumor suppressor gene promotes hyaloid vascular regression during mouse eye development. Proc Natl Acad Sci USA. 99:3848-53.
35. Meek D W. 2009. Tumor suppression by p53: a role for the DNA damage response? Nat Rev Cancer. 9:714-23.
36. Mooi W J. 2009. Oncogene-induced cellular senescence: causal factor in the growth arrest of pituitary microadenomas? Horm Res. 71 Suppl 2:78-81.
37. Nishino J, Kim I, Chada K, Morrison S J. 2008. Hmga2 promotes neural stem cell self-renewal in young but not old mice by reducing p16Ink4a and p19Arf expression. Cell. 135:227-39.
38. Ono M, Maruyama T, Masuda H, Kajitani T, Nagashima T, Arase T, Ito M, Ohta K, Uchida H, Asada H, Yoshimura Y, Okano H, Matsuzaki Y. 2007. Side population in human uterine myometrium displays phenotypic and functional characteristics of myometrial stem cells. Proc Natl Acad Sci USA. 104:18700-5
39. Rein M S, Powell W L, Walters F C, Weremowicz S, Cantor R M, Barbieri R L, Morton C C. 1998. Cytogenetic abnormalities in uterine myomas are associated with myoma size. Mol Hum Reprod. 4:83-6.
40. Romagnolo B, Molina T, Leroy G, Blin C, Porteux A, Thomasset M, Vandewalle A, Kahn A, Perret C. 1996. Estradiol-dependent uterine leiomyomas in transgenic mice. J Clin Invest. 98:777-84.
41. Sandberg A A. 2005. Updates on the cytogenetics and molecular genetics of bone and soft tissue tumors: leiomyoma. Cancer Genet Cytogenet. 158:1-26.
42. Schoenmakers E F, Wanschura S, Mols R, Bullerdiek J, Van den Berghe H, Van de Ven W J. 1995. Recurrent rearrangements in the high mobility group protein gene, HMGI-C, in benign mesenchymal tumors. Nat. Genet. 10:436-44.
43. Sebastian T, Johnson P F. 2009. RasV12-mediated down-regulation of CCAAT/enhancer binding protein beta in immortalized fibroblasts requires loss of p19Arf and facilitates bypass of oncogene-induced senescence. Cancer Res. 69:2588-98.
44. Serrano M, Lee H, Chin L, Cordon-Cardo C, Beach D, DePinho R A. 1996. Role of the INK4a locus in tumor suppression and cell mortality. Cell. 85:27-37
45. Shynlova O, Tsui P, Jaffer S, Lye S J. 2009. Integration of endocrine and mechanical signals in the regulation of myometrial functions during pregnancy and labour. Eur J Obstet Gynecol Reprod Biol. 144:2-10
46. Specht K, Richter T, Miller U, Watch A, Werner M, Holler H.2001. Quantitative gene expression analysis in microdissected archival formalin-fixed and paraffin-embedded tumor tissue. Am J. Pathol. 158:419-29.
47. Utikal J, Polo J M, Stadtfeld M, Maherali N, Kulalert W, Walsh R M, Khalil A, Rheinwald J G, Hochedlinger K. 2009 Immortalization eliminates a roadblock during cellular reprogramming into iPS cells. Nature. 460:1145-8.
48. Webster M A, Martin-Soudant N, Nepveu A, Cardiff R D, Muller W J. 1998. The induction of uterine leiomyomas and mammary tumors in transgenic mice expressing polyomavirus (PyV) large T (LT) antigen is associated with the ability of PyV LT antigen to form specific complexes with retinoblastoma and CUTL1 family members. Oncogene. 16:1963-72.
49. Wei J J, Soteropoulos P. 2008. MicroRNA: a new tool for biomedical risk assessment and target identification in human uterine leiomyomas. Semin Reprod Med. 26:515-21.
50. Zhang Y, Xiong Y, Yarbrough W G. 1998. ARF promotes MDM2 degradation and stabilizes p53: ARF-INK4a locus deletion impairs both the Rb and p53 tumor suppression pathways. Cell. 92:725-34.
51. Gorzelniak K, Janke J, Engeli S, Sharma A M. 2001. Validation of endogenous controls for gene expression studies in human adipocytes and preadipocytes. Horm. Metab Res. 33:625-7.
52. Bullwinkel J, Baron-Lühr B, Lüdemann A, Wohlenberg C, Gerdes J, Scholzen T. 2006. Ki-67 protein is associated with ribosomal RNA transcription in quiescent and proliferating cells. J Cell Physiol. 206:624-35.
53. Scholzen T, Gerdes J. 2000. The Ki-67 protein: from the known and the unknown. J Cell Physiol. 182:311-22.
54. Miao Y, Cui T, Leng F, Wilson W D. 2008 Inhibition of high-mobility-group A2 protein binding to DNA by netropsin: a biosensor-surface plasmon resonance assay. Anal Biochem. 374:7-15.
55. Xiong, Y., H. Zhang, and D. Beach. 1993. Subunit rearrangement of the cyclin-dependent kinases is associated with cellular transformation. Genes & Dev. 7: 1572-1583.
56. Di Leonardo, A., S. P. Linke, K. Clarkin, and G. M. Wahl. 1994. DNA damage triggers a prolonged p53-dependent G1 arrest and long-term induction of Cip1 in normal human fibroblasts. Genes & Dev. 8: 2540-2551.
57. El-Diery, W. S., T. Tokino, V. E. Velculescu, D. B. Levy, R. Parsons, J. M. Trent, D. Lin, W. E. Mercer, K. Kinzler, and B. Vogelstein. 1993. WAF1, a potential mediator of p53 tumor suppression. Cell 75:817-825.
58. E1-Deiry, W., J. W. Harper, P. M. O'Connor, V. E. Velculescu, C. E. Canman, J. Jackman, J. A. Pietenpol, M. Burrell, D. E. Hill, Y. Wang, K. G. Wiman, W. E. Mercer, M. B. Kastan, K. W. Kohn, S. J. Elledge, K. W. Kinzler, and B. Vogelstein. 1994. WAF1/CIP1 is induced in p53-mediated G1 arrest and apoptosis. Cancer Res. 54:1169-1174.
59. Efeyan A, Ortega-Molina A, Velasco-Miguel S, Herranz D, Vassilev L T, Serrano M. 2007. Induction of p53-dependent senescence by the MDM2 antagonist nutlin-3a in mouse cells of fibroblast origin. Cancer Res. 67:7350-7.
60. Brenner, R. M., and Maslar, I. A. 1988 The primate oviduct and endometrium. In The Physiology of Reproduction (E. Knobil and J. Neill, Edsl) Vol. 1 Raven Press, New York)
61. Langman, J. 1979. Medical Embryology, Williams & Wilkins, Baltimore
62. Kurita, T., Cooke, P. S., Cunha, G. R. 2001. Epithelial-stromal tissue interaction in paramesonephric (Mullerian) epithelial differentiation. Dev Biol 240, 194-211.
63. Teixeira, J., Rueda, B. R., and Pru, J. K. 2008. Uterine Stem cells, StemBook, ed. The Stem Cell Research Community, StemBook, doi/10.3824/stembook.1.16.1, http://www.stembook.org,
64. Cook, A. and Cowan, C. 2009. Adipose (March 31,), StemBook, ed. The Stem Cell Research Community, StemBook, doi/10.3824/stembook.1.40.1
65. Schipani, E., and Kronenberg, H. M. 2009. Adult mesenchymal stem cells, StemBook, ed. The Stem Cell Research Community, StemBook, doi/10.3824/stembook.1.38.1

Further literature concerning any one of the materials, methods, uses and compounds to be employed in accordance with the present invention may be retrieved from public libraries and databases, using for example electronic devices. For example the public database “Medline” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks, TIBTECH 12 (1994), 352-364.

1. A method of treating a subject having uterine leiomyoma (UL), comprising administering an MDM2 inhibitor selected from nutlins in a therapeutically effective amount to a patient in need thereof.

2. The method of claim 1, wherein the MDM2 inhibitor is administered by oral dosage administration.

3. The method of claim 2, wherein the oral dosage administration of the MDM2 inhibitor is repeated.

4. The method of claim 3, wherein the administration of the oral dosage of the MDM2 inhibitor is repeated once a year.

5. The method of claim 1, wherein the MDM2 inhibitor is administered by local administration.

6. The method of claim 5, wherein the local administration of the MDM2 inhibitor is repeated.

7. The method of claim 6, wherein the local administration of the MDM2 inhibitor is repeated once a year.

8. The method of claim 1, wherein the MDM2 inhibitor is nutlin-3.

9. The method of claim 2, wherein the oral dosage administration of the MDM2 inhibitor is combined with other medical therapies comprising the administration of non-steroidal anti-inflammatory drugs, GnRH (gonadotropin-releasing hormone) agonists, GnRH antagonists, and progesterone-releasing intrauterine devices.

10. The method of claim 5, wherein the local administration of the MDM2 inhibitor is combined with other medical therapies comprising the administration of non-steroidal anti-inflammatory drugs, GnRH (gonadotropin-releasing hormone) agonists, GnRH antagonists, and progesterone-releasing intrauterine devices.

11. The method of claim 1, wherein the nutlins are selective for uterine leiomyoma tissue in comparison to surrounding myometrial tissue.

12. The method of claim 11, wherein uterine leiomyoma tissue is more sensitive to the apoptotic and senescent effects of nutlins in comparison to surrounding myometrial tissue.

13. The method of claim 1, wherein the MDM2 inhibitor is designed to be administered in a pharmaceutical composition.

14. The method of claim 13, wherein the pharmaceutical composition additionally comprises an acceptable carrier.
